(19) United States
(12) Patent Application Publication
(10) Pub. No.: US 2025/0213484 A1
(43) Pub. Date: Jul. 3, 2025

(54) A FREEZE-DRYING PROCESS FOR AN ADC

(71) Applicant: GENEQUANTUM HEALTHCARE (SUZHOU) CO., LTD.

(72) Inventors: Gang QIN, Suzhou (CN); Bian GUO, Suzhou (CN); Xuesong LI, Suzhou (CN); Lili SHI, Suzhou (CN); Cao LV, Suzhou (CN)

(21) Appl. No.: 18/851,893

(22) PCT Filed: Mar. 29, 2023
(86) PCT No.: PCT/CN2023/084611

§ 371 Date: Sep. 27, 2024

(30) Foreign Application Priority Data
Mar. 29, 2022 (WO) PCT/CN2022/083700

(51) Int. Cl. A61K 9/19; A61K 31/5355; A61K 47/68

(52) U.S. Cl. CPC A61K 9/19; A61K 31/5355; A61K 47/6803; A61K 47/6849

(57) ABSTRACT

Provided is a freeze-drying process for an antibody drug conjugate, wherein the ADC is GQ1001. Due to the use of the present freeze-drying process parameters, especially the suitable pre-freezing cooling rate and a suitable drying vacuum, the product obtained by the process has many advantages in physical and chemical properties.

This application claims the priority to PCT/CN2022/083700 filed on Mar. 29, 2022, which is incorporated herein by reference in its entirety.

### FIELD OF THE INVENTION

Provided is a freeze-drying process for an antibody drug conjugate, which belongs to the field of pharmaceutical production.

### BACKGROUND OF THE INVENTION

GQ1001 is a site-specific anti-HER2 antibody-drug conjugate (ADC) developed using a new generation of antibody conjugation technology. Compared with the prior ADC drugs such as T-DM1 using chemical conjugation technology, GQ1001 realizes the site-specific and quantitative connection of small molecule cytotoxins on antibodies, which is highly homogeneous and stable, and has greatly improved quality characteristics and therapeutic index. Thus, it has great clinical value and market prospect. Freeze-drying is widely used in the pharmaceutical industry, food industry, scientific research and other sectors because of its many advantages. For example, freeze-drying is carried out at low temperature, so it is particularly suitable for many heat sensitive substances; in the process of freeze-drying, the growth of microorganisms and the action of enzymes cannot be carried out, so the original properties can be maintained; the dried substance dissolves rapidly and completely after adding water, and almost immediately recovers its original properties; drying can remove more than 95-99% water, so that the dried products can be stored for a long time without deterioration.

The quality of freeze-dried products is related to many factors, including the nature of the product itself, pre-freezing temperature, cooling rate, heating rate, drying temperature, vacuum degree, drying time, etc. Freeze-drying is generally divided into two stages, pre-freezing and drying. Freezing may destroy cells and life bodies, which is generally considered to be mainly caused by the mechanical effect and solute effect. The mechanical effect and solute effect are closely related to the cooling rate during pre-freezing. For the purpose of improving cell survival rate or reducing protein denaturation during freeze-drying, an appropriate cooling rate is needed for pre-freezing. In order to freeze dry a good product, a good and stable vacuum degree in the drying process is also essential. Low pressure is conducive to the sublimation of ice in the product, but too low pressure is unfavorable to heat transfer, the product is not easy to obtain heat, and the sublimation rate decreases. Therefore, the selection of drying pressure is very important for freeze-drying process.

Freeze-dried products of antibody-drug conjugates (ADCs) need to have a certain physical form, uniform color, qualified residual moisture content, good solubility, high potency and long-term storage. In order to achieve the above-mentioned better product effects and increase production capacity, an optimized freeze-drying process is very necessary.

Different products often need different appropriate freeze-drying processes. The invention adopts an improved freeze-drying process to obtain an antibody-drug conjugate product with good appearance and stable properties and improve production efficiency. After the product reconstitution, there are no significant changes in the protein concentration, pH, DAR values, the purity of SEC-HPLC, the purity of CE-SDS, the charge variants of CEX-HPLC. And the free drug and moisture content are still low, and the biological activity and binding activity are still within the range of 100±30%. Additionally, the product has a good light stability.

### SUMMARY

In one aspect, provided is a freeze-drying process of an ADC formulation, comprising the following steps:
(1) Pre-freezing:
transferring the ADC formulation to the freezer layer of a lyophilizer,
lowering the temperature with a cooling rate of about 0.36° C./min or more;
(2) Primary drying:
raising the temperature and drying under the pressure of about 30 Pa or less;
(3) Secondary drying:
raising the temperature to about 20-40° C.;
wherein,
step (1) comprises a cooling process and a holding process;
step (2) comprises a heating process and a holding process;
step (3) comprises a heating process and a holding process;
wherein,
the ADC is GQ1001 having the structure of

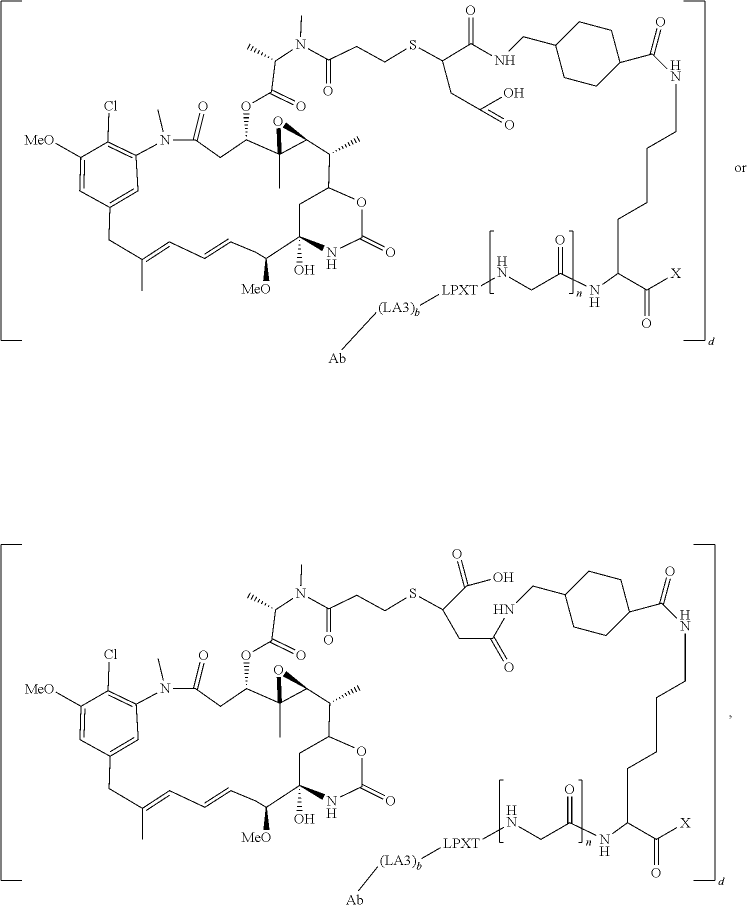

wherein n is 3, d is 2, the X in the ligase recognition sequence LPXT is a glutamic acid (E), Ab is Trastuzumab, LA3 is linker moiety, comprising 1 to 100 series-connected structure units which are selected from the group consisting of one or more glycine and alanine; each b is independently 0 or 1, indicating the presence or absence of LA3; x is —OH or —NH₂ group; preferably, the ADC is

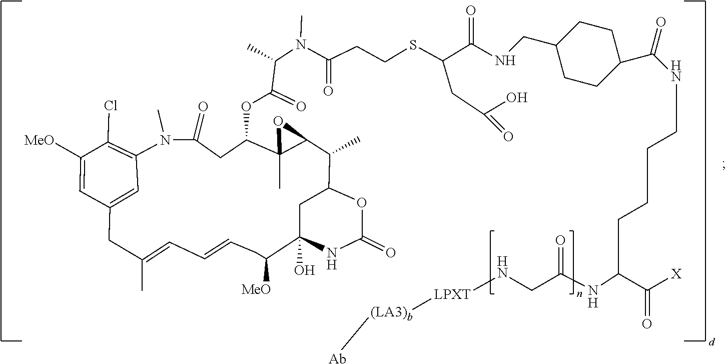

wherein,
the ADC formulation comprises
15-25 mg/ml GQ1001,
8-12 mmol/L sodium succinate,
4-8% sucrose (W/V),
0.01-0.03% polysorbate 20 (W/V), and
pH is 4.8-5.2.

In another aspect, provided could be used in a full-tank freeze-drying process.

### DRAWINGS

FIG. 1 shows the freeze-drying curve of a small-scale test sample.

FIG. 2 shows the freeze-drying curve (1° C./min) of the pre-freeze sublimation rate test 1.

FIG. 3 shows the freeze-drying curve (0.3° C./min) of the pre-freeze sublimation rate test 2.

FIG. 4 shows a freeze-drying curve (−25° C./25 Pa) of primary drying sublimation rate test 1.

FIG. 5 shows a freeze-drying curve (−20° C./35 Pa) of primary drying sublimation rate test 2.

FIG. 6 shows a freeze-drying curve (−20° C./25 Pa) of primary drying sublimation rate test 3.

FIG. 7 shows a freeze-drying curve (−25° C./35 Pa) of primary drying sublimation rate test 4.

FIG. 8 shows a complete freeze-drying curve (−20° C./35 Pa) of primary drying test 1.

FIG. 9 shows a complete freeze-drying curve (−20° C./25 Pa) of primary drying test 2.

FIG. 10 shows the optimized freeze-drying curve (30° C./8 h) of the secondary drying.

FIG. 11 shows a freeze-drying curve confirmed by freeze-drying optimization.

FIG. 12 shows the freeze-drying curve of the lyophilized enlarged full-tank prepared Buffer.

FIG. 13 shows the freeze-drying curve of the process development confirmation batch sample preparation.

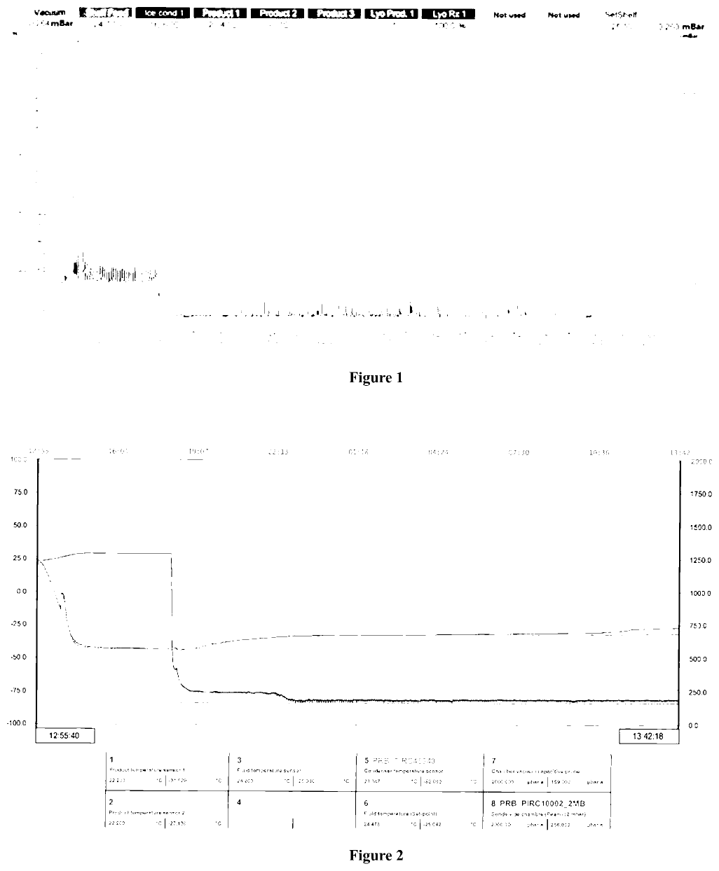

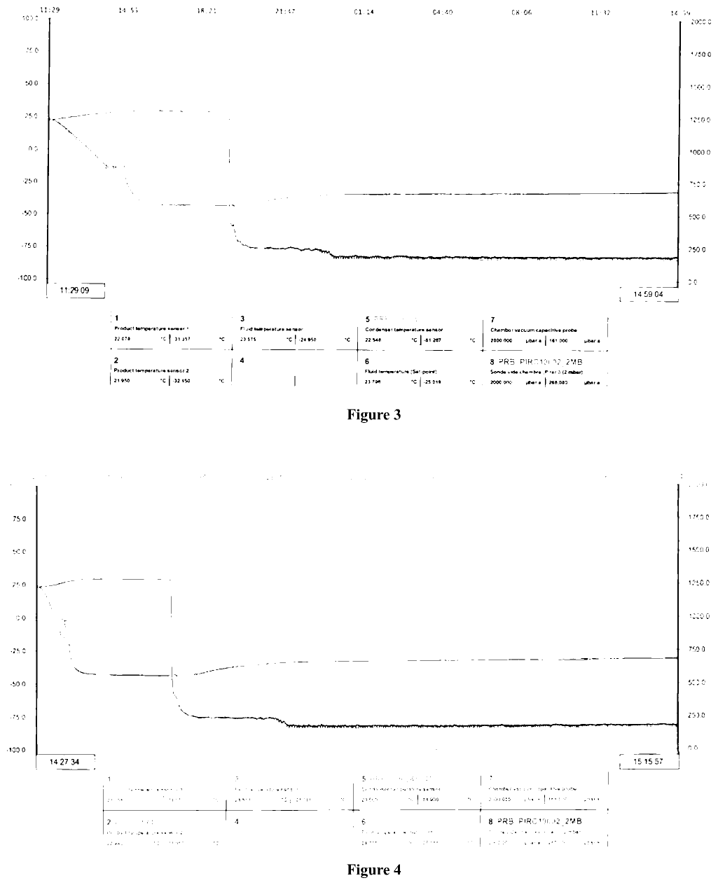

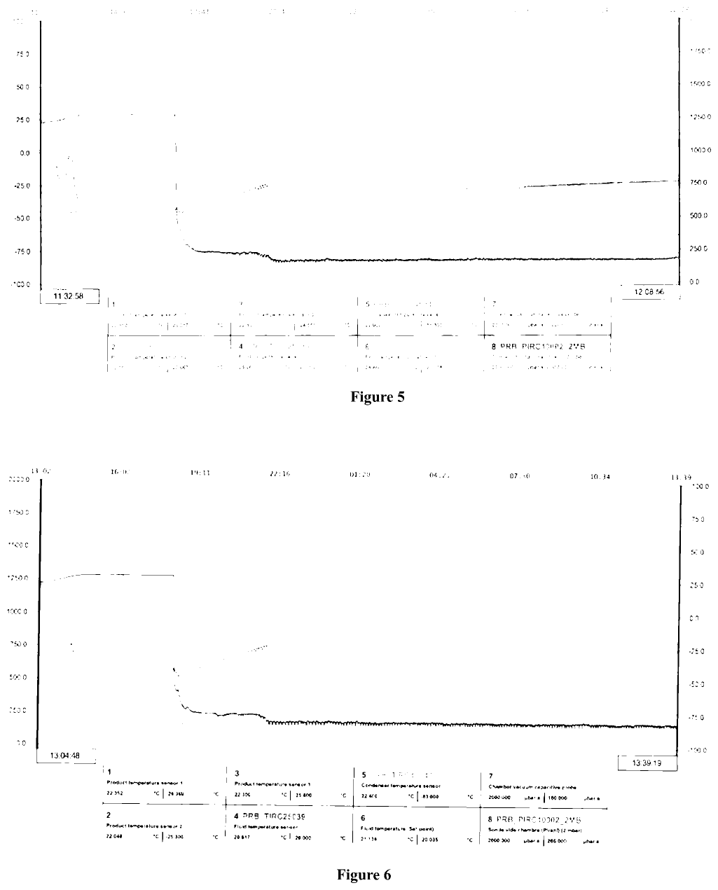

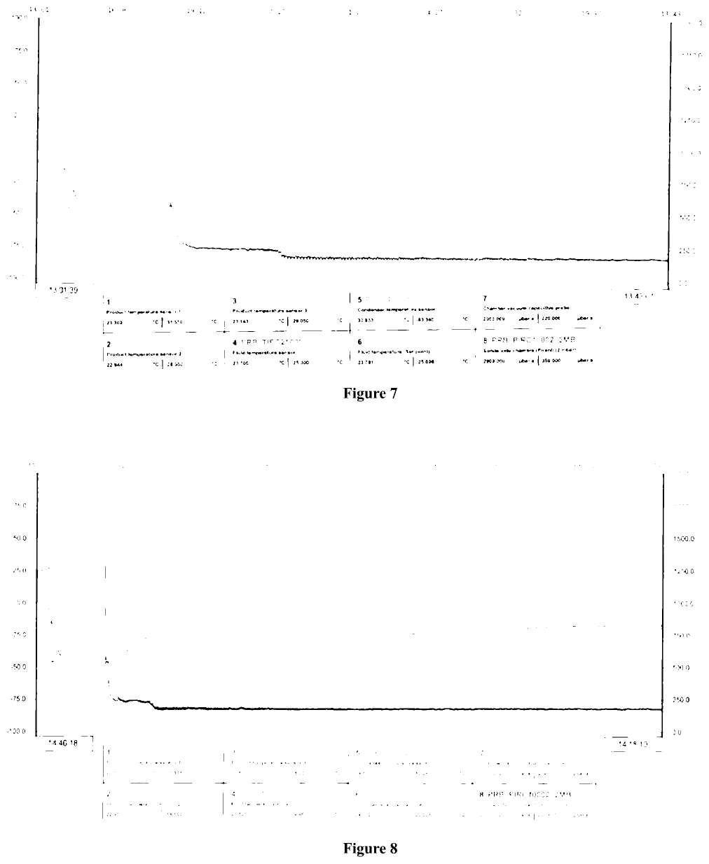

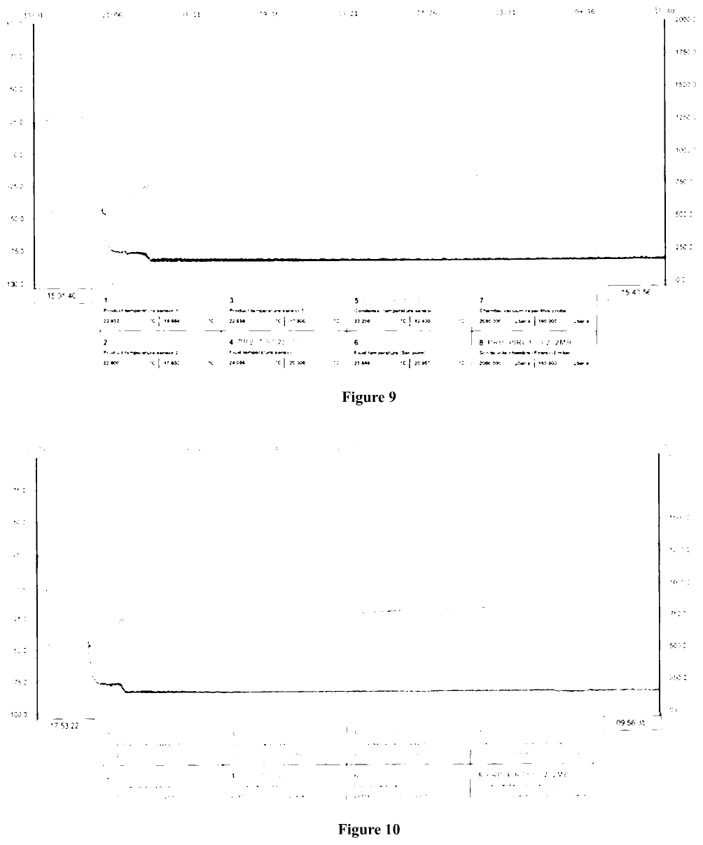

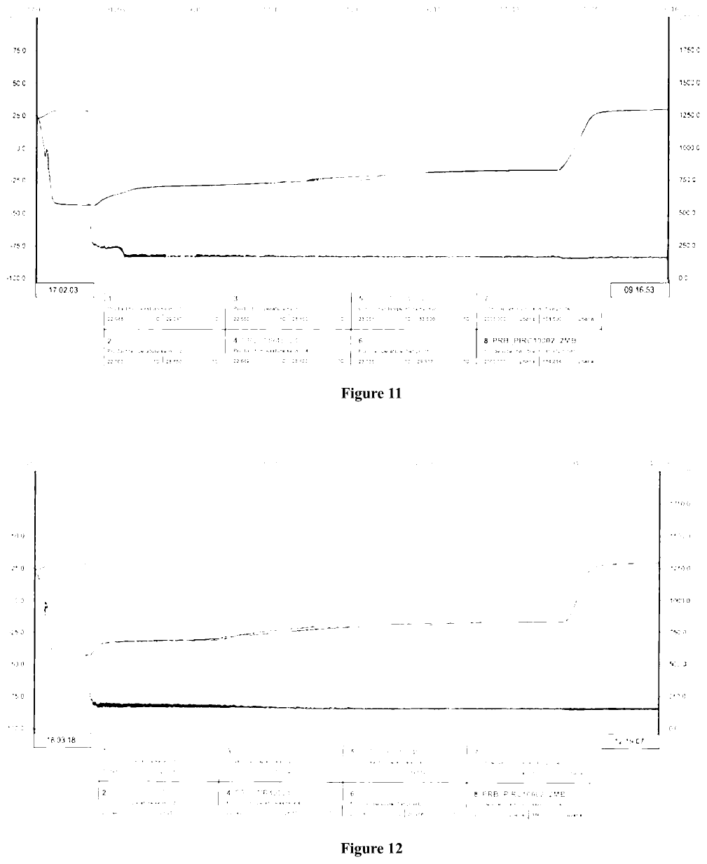

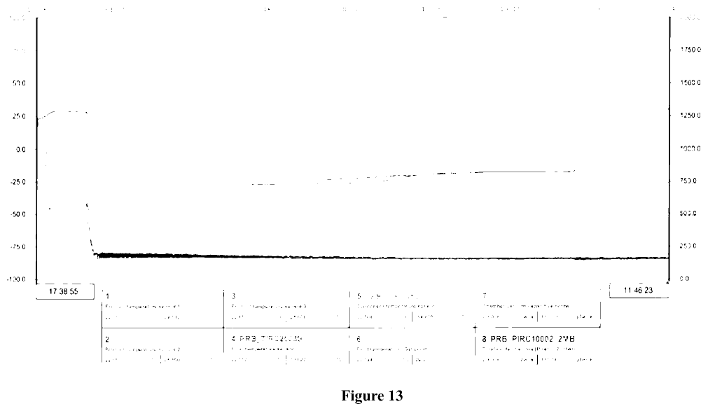

### DETAILED DESCRIPTION

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “20-40° C.” means any temperature of 20 to 40° C., for example, it can be 20, 21, 22, 23, 24, 25, 26, 27, 28, 29, 30, 31, 32, 33, 34, 35, 36, 37, 38, 39 or 40° C.; the expression “4 hours or more” means any time of no less than 4 hours, for example, it can be 4, 5, 6, 7, 8, 9, 10 or more hours. the expression “10-30° C./h” means any temperature rate of 10 to 30° C./h, for example, it can be 10, 11, 12, 13, 14, 15, 16, 17, 18, 19, 20, 21, 22, 23, 24, 25, 26, 27, 28, 29 or 30° C./h. Other similar expressions should also be understood in a similar manner.

Unless otherwise stated herein, singular forms like “a” and “the” include the plural forms.

The term “antibody-drug conjugate (ADC)” refers to the connection of a small molecule drug with biological activity to a monoclonal antibody through a chemical link. The monoclonal antibody acts as a carrier to target the small molecule drug to the target cell. Its main components include antibody, linker and small molecular cytotoxic drug (SM).

The term “GQ1001” refers to an ADC having the following structure:

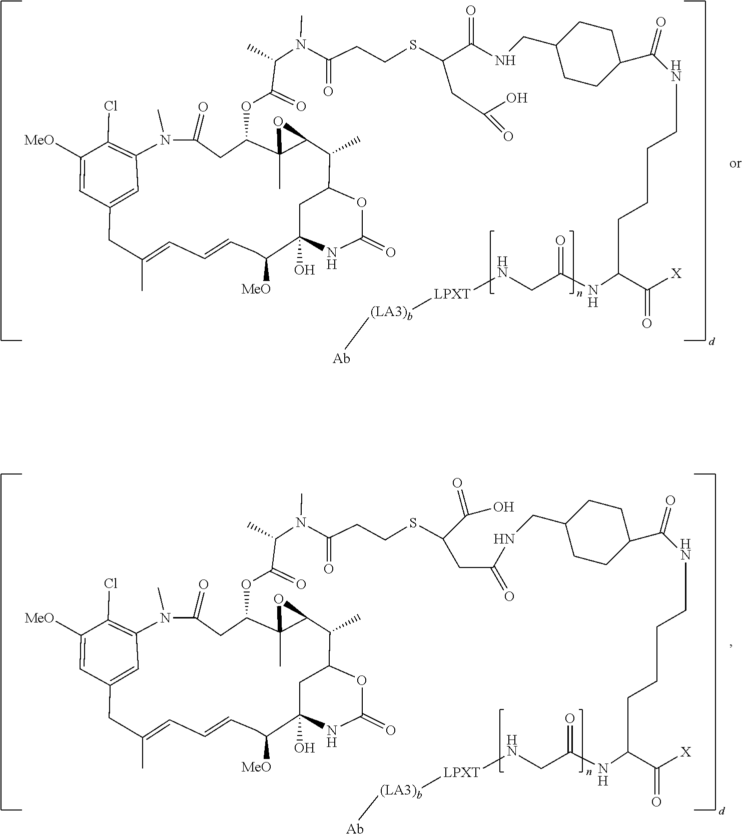

wherein n is 3, d is 2, the X in the ligase recognition sequence LPXT is a glutamic acid (E), Ab is Trastuzumab, LA3 is linker moiety, comprising 1 to 100 series-connected structure units which are selected from the group consisting of one or more glycine and alanine; each b is independently 0 or 1, indicating the presence or absence of LA3; x is —OH or —NH₂ group. Relevant information regarding the ADC molecule can also be found in EP3138568 B1, which is incorporated herein by reference.

The term “freeze-drying” or “lyophilization” refers to a drying method in which water-containing materials are frozen below the freezing point, water is converted into ice, and then the ice is converted into vapor under a relatively high vacuum to be removed.

The term “freeze-drying curve” refers to the relationship curve that represents the temperature and pressure of the product during the freeze-drying process with time.

The term “pre-freezing” refers to the process of freezing products that contain a large amount of moisture into solids.

The term “normal pressure” refers to a standard atmospheric pressure, namely 101325 Pa.

The term “drying” refers to the process of separating moisture from the product.

The terms “about”, when used in connection with a numerical variable, generally mean that the value of the variable and all values of the variable are within experimental error (for example, within a 95% confidence interval for the mean) or within ±10% of a specified value, or a wider range.

The expression “comprising” or similar expressions “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

In an embodiment, the freeze-drying process of ADC includes the following steps:
(1) Pre-freezing:
transferring the ADC formulation to the freezer layer of a lyophilizer,
lowering the temperature with a cooling rate of about 0.36° C./min or more;
(2) Primary drying:
raising the temperature and drying under the pressure of about 30 Pa or less;
(3) Secondary drying:
raising the temperature to about 20-40° C.

In an embodiment, the ADC formulation of the freeze-drying process is a GQ1001 formulation, wherein the GQ1001 has the structure of

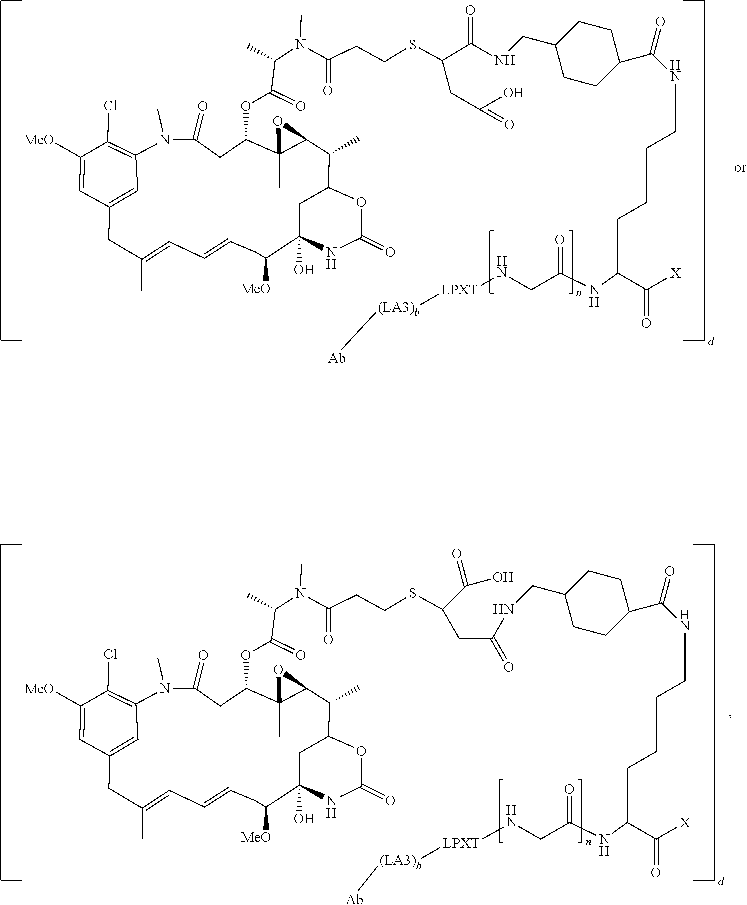

wherein n is 3, d is 2, the X in the ligase recognition sequence LPXT is a glutamic acid (E), Ab is Trastuzumab, LA3 is linker moiety, comprising 1 to 100 series-connected structure units which are selected from the group consisting of one or more glycine and alanine; each b is independently 0 or 1, indicating the presence or absence of LA3; x is —OH or —NH₂ group.

In an embodiment, the GQ1001 has the structure of

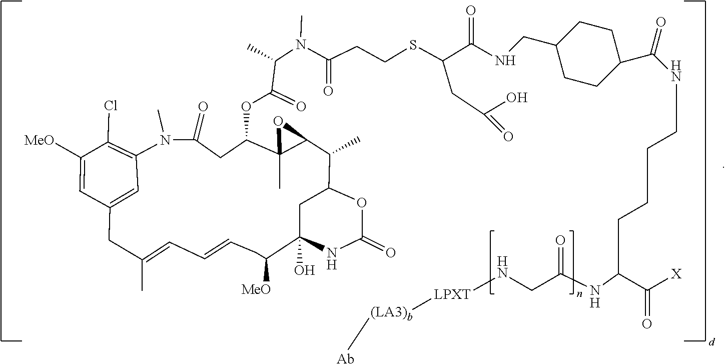

In an embodiment, the ADC GQ1001 formulation comprises 15-25 mg/ml GQ1001, preferably 20 mg/ml.

In an embodiment, the ADC GQ1001 formulation comprises 8-12 mmol/L sodium succinate, preferably 10 mmol/L.

In an embodiment, the ADC GQ1001 formulation comprises 4-8% sucrose (W/V), preferably 6% sucrose (W/V).

In an embodiment, the ADC GQ1001 formulation comprises 0.01-0.03% polysorbate 20 (W/V), preferably 0.02% polysorbate 20 (W/V).

In an embodiment, the pH of the ADC GQ1001 formulation is 4.8-5.2, preferably 5.0.

In an embodiment, the ADC GQ1001 formulation comprises 15-25 mg/ml GQ1001, 8-12 mmol/L sodium succinate, 4-8% sucrose (W/V), 0.01-0.03% polysorbate 20 (W/V), and pH is 4.8-5.2.

In a specific embodiment, the ADC GQ1001 formulation comprises 20 mg/ml GQ1001, 10 mmol/L sodium succinate, 6% sucrose (W/V), 0.02% polysorbate 20 (W/V), and the pH is 5.0.

In an embodiment, the step (1) of the freeze-drying process comprises a cooling process and a holding process; step (2) comprises a heating process and a holding process; step (3) comprises a heating process and a holding process.

In an embodiment, the freeze-drying process of an ADC formulation comprises the following steps:
(1) Pre-freezing:
transferring the ADC formulation to the freezer layer of a lyophilizer,
lowering the temperature with a cooling rate of about 0.36° C./min or more;
(2) Primary drying:
raising the temperature and drying under the pressure of about 30 Pa or less;
(3) Secondary drying:
raising the temperature to about 20-40° C.;
wherein,
step (1) comprises a cooling process and a holding process;
step (2) comprises a heating process and a holding process;
step (3) comprises a heating process and a holding process,
wherein,
the ADC is GQ1001 having the structure of

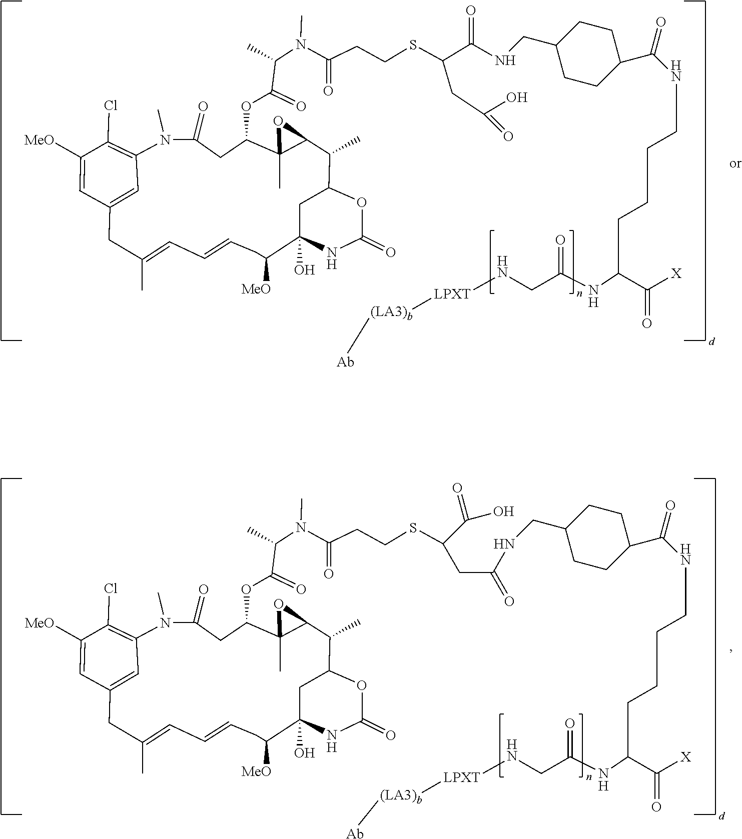

wherein n is 3, d is 2, the X in the ligase recognition sequence LPXT is a glutamic acid (E), Ab is Trastuzumab, LA3 is linker moiety, comprising 1 to 100 series-connected structure units which are selected from the group consisting of one or more glycine and alanine; each b is independently 0 or 1, indicating the presence or absence of LA3; x is —OH or —NH₂ group; preferably, the ADC is

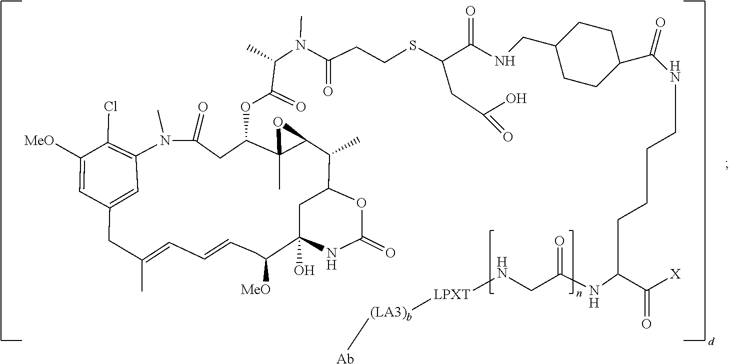

wherein,
the ADC formulation comprises
15-25 mg/ml GQ1001,
8-12 mmol/L sodium succinate,
4-8% sucrose (W/V),
0.01-0.03% polysorbate 20 (W/V), and
pH is 4.8-5.2.

In an embodiment, the initial temperature of ADC formulation in step (1) is about 18-22° C., preferably 20° C.

In an embodiment, the temperature of the holding process in step (1) is about −45° C. or less.

In an embodiment, the temperature of the holding process in step (1) is no less than about −60° C.

In an embodiment, the temperature of the holding process in step (1) is about −45° C.

In an embodiment, the cooling rate of the cooling process in step (1) is no more than about 2° C./min.

In an embodiment, the cooling rate of the cooling process in step (1) is about 0.36-1.5° C./min.

In an embodiment, the cooling rate of the cooling process in step (1) is about 0.37° C./min.

In an embodiment, the cooling rate of the cooling process in step (1) is about 0.5° C./min or more.

In a specific embodiment, the cooling rate of the cooling process in step (1) is about 1° C./min.

In another specific embodiment, the cooling rate of the cooling process in step (1) is about 1.08° C./min.

In an embodiment, the cooling time in step (1) is about 3 hours or less.

In an embodiment, the cooling time in step (1) is about 1-2 hours.

In a specific embodiment, the cooling time in step (1) is about 1 hour.

In an embodiment, the holding time in step (1) is about 4 hours or more.

In an embodiment, the holding time in step (1) is about 4-10 hours.

In an embodiment, the holding time in step (1) is about 8 hours.

In a specific embodiment, the holding time in step (1) is about 4 hours.

In an embodiment, the pressure of the freeze-drying chamber during the cooling process in step (1) is normal pressure.

In an embodiment, the pressure of the freeze-drying chamber during the holding process in step (1) is normal pressure.

In a specific embodiment, the pressure of the freeze-drying chamber during step (1) is normal pressure.

In an embodiment, the increase temperature rate of the heating process in step (2) is about 5-26° C./h.

In an embodiment, the increase temperature rate of the heating process in step (2) is about 5° C./h.

In an embodiment, the increase temperature rate of the heating process in step (2) is about 6-25° C./h.

In a specific embodiment, the increase temperature rate of the heating process in step (2) is about 6° C./h.

In another specific embodiment, the increase temperature rate of the heating process in step (2) is about 25° C./h.

In an embodiment, the increase temperature time of the heating process in step (2) is about 5 hours or less.

In a specific embodiment, the increase temperature time of the heating process in step (2) is about 4 hours.

In another specific embodiment, the increase temperature time of the heating process in step (2) is about 1 hour.

In an embodiment, the pressure of the freeze-drying chamber during the heating process in step (2) is about 20-30 Pa.

In a specific embodiment, the pressure of the freeze-drying chamber during the heating process in step (2) is about 25 Pa.

In an embodiment, the pressure of the freeze-drying chamber during the heating process in step (2) is no less than about 20 Pa. In an embodiment, the temperature of holding process in step (2) is about −26 to ˜18° C.

In an embodiment, the temperature of holding process in step (2) is about −25 to −20° C.

In an embodiment, the temperature of holding process in step (2) is about −25° C.

In a specific embodiment, the temperature of holding process in step (2) is about −20° C.

In an embodiment, the holding time in step (2) is about 10-55 hours.

In an embodiment, the holding time in step (2) is about 15 hours.

In an embodiment, the holding time in step (2) is about 40-49 hours.

In an embodiment, the holding time in step (2) is about 40 hours.

In a specific embodiment, the holding time in step (2) is about 43 hours.

In another specific embodiment, the holding time in step (2) is about 49 hours.

In an embodiment, the pressure of the freeze-drying chamber during the holding process in step (2) is about 20-30 Pa.

In an embodiment, the pressure of the freeze-drying chamber during the holding process in step (2) is about 25 Pa.

In an embodiment, the pressure of the freeze-drying chamber during the holding process in step (2) is no less than 20 Pa.

In an embodiment, the pressure of the freeze-drying chamber during step (2) is about 20-30 Pa.

In a specific embodiment, the pressure of the freeze-drying chamber during step (2) is about 25 Pa.

In an embodiment, the pressure of the freeze-drying chamber during step (2) is no less than 20 Pa.

In an embodiment, the increase temperature rate of the heating process in step (3) is about 10-30° C./h.

In an embodiment, the increase temperature rate of the heating process in step (3) is about 10° C./h.

In a specific embodiment, the increase temperature rate of the heating process in step (3) is about 17° C./h.

In another specific embodiment, the increase temperature rate of the heating process in step (3) is about 18° C./h.

In yet another specific embodiment, the increase temperature rate of the heating process in step (3) is about 25° C./h.

In an embodiment, the increase temperature time in step (3) is about 2-6 hours.

In an embodiment, the increase temperature time in step (3) is about 5 hours.

In an embodiment, the increase temperature time in step (3) is about 2-4 hours.

In a specific embodiment, the increase temperature time in step (3) is about 2 hours.

In another specific embodiment, the increase temperature time in step (3) is about 3 hours.

In an embodiment, the pressure of the freeze-drying chamber during the heating process in step (3) is about 20-30 Pa.

In a specific embodiment, the pressure of the freeze-drying chamber during the heating process in step (3) is about 25 Pa.

In an embodiment, the pressure of the freeze-drying chamber during the heating process in step (3) is no less than about 20 Pa.

In an embodiment, the temperature of the holding process in step (3) is about 20-35° C.

In an embodiment, the temperature of the holding process in step (3) is about 25-30° C.

In a specific embodiment, the temperature of the holding process in step (3) is about 25° C.

In another specific embodiment, the temperature of the holding process in step (3) is about 30° C.

In an embodiment, the holding time in step (3) is about 6 hours or more.

In a specific embodiment, the holding time in step (3) is about 8 hours.

In another specific embodiment, the holding time in step (3) is about 10 hours.

In an embodiment, the pressure of the freeze-drying chamber during the holding process in step (3) is about 20-30 Pa.

In an embodiment, the pressure of the freeze-drying chamber during the holding process in step (3) is about 25 Pa.

In an embodiment, the pressure of the freeze-drying chamber during the holding process in step (3) is no less than about 20 Pa.

In an embodiment, the pressure of the freeze-drying chamber during step (3) is about 20-30 Pa.

In a specific embodiment, the pressure of the freeze-drying chamber during step (3) is about 25 Pa.

In an embodiment, the pressure of the freeze-drying chamber during step (3) is no less than about 20 Pa.

In an embodiment, the pressure of the freeze-drying chamber during step (2) and step (3) is about 20-30 Pa.

In a specific embodiment, the pressure of the freeze-drying chamber during step (2) and step (3) is about 25 Pa.

In an embodiment, the pressure of the freeze-drying chamber during step (2) and step (3) is no less than about 20 Pa.

In an embodiment, the increase temperature rate of step (2) is about 6° C./h, the increase temperature time of step (2) is about 4 hours, and the holding time of step (2) is about 43 hours.

In an embodiment, the increase temperature of step (2) is about 25° C./h, the increase temperature time of step (2) is about 1 hour, and the holding time of step (2) is about 49 hours.

In an embodiment, the increase temperature rate of step (3) is about 17° C./h, the increase temperature time of step (3) is about 3 hours, and the holding time of step (3) is about 8 hours.

In an embodiment, the increase temperature rate of step (3) is about 25° C./h, the increase temperature time of step (3) is about 2 hours, and the holding time of step (3) is about 8 hours.

In an embodiment, the temperature of the holding process in step (1) is −45° C., the temperature of the holding process in step (2) is about −20° C., and the temperature of the holding process in step (3) is about 25° C.

In an embodiment, the temperature of the holding process in step (1) is −45° C., the temperature of the holding process in step (2) is about −20° C., and the temperature of the holding process in step (3) is about 30° C.

In an embodiment, the cooling rate of step (1) is about 65° C./h, the increase temperature rate of step (2) is about 6° C./h, and the increase temperature rate of step (3) is about 17° C./h.

In an embodiment, the cooling rate of step (1) is about 1° C./min, the increase temperature rate of step (2) is about 25° C./h, and the increase temperature rate of step (3) is about 25° C./h.

In an embodiment, the freeze-drying process of ADC formulation is used for full-tank freeze-drying.

In an embodiment, the volume of full-tank freeze-drying is about 0.4 m² or more, preferably about 0.5 m² or more.

In a specific embodiment, the volume of full-tank freeze-drying is about 0.5 m².

In an embodiment, the volume of the full-tank freeze-drying is no more than about 2 m².

In an embodiment, the temperature of pre-freezing is determined by the collapse temperature and glass state transition temperature of the protein stock solution and formulation buffer, respectively.

In an embodiment, the parameters used for determining the stability of ADC formulation comprises one or more of appearance/color, pH, osmotic pressure, clarity, protein concentration, insoluble sub-visible particles, DAR value, the proportion of the main peak of SEC-HPLC of the sample, the purity of CE-SDS, the proportion of the main peak of CEX-HPLC, the free drug, moisture content, the binding activity and biological activity.

In an embodiment, the loading amount of the antibody-drug conjugate is 5.3 ml/vial.

In an embodiment, the product obtained by the freeze-drying process has a reconstitution time of less than 1 minute.

In an embodiment, the biological activity and binding activity of the product obtained by the freeze-drying process are within the range of 100±30% after reconstitution.

In an embodiment, the product obtained by the freeze-drying process has a good light stability.

In an embodiment, the product obtained by the freeze-drying process has a good protein stability after reconstitution.

### Beneficial Effects

The present invention provides a freeze-drying process for an ADC formulation, wherein the ADC is GQ1001. Due to the use of the present freeze-drying process parameters, especially the suitable pre-freezing cooling rate and a suitable drying vacuum, the freeze-drying time is reduced while ensuring that the freeze-dried product avoids collapse and maintains a good product appearance, thereby improving the output efficiency of the product.

The product of the present freeze-drying process has low moisture content and particle level and has a reconstitution time of less than 1 minute. After the product reconstitution, there are no significant changes in the protein concentration, pH, DAR values, the purity of SEC-HPLC, the purity of CE-SDS, the proportion of charge variants of CEX-HPLC. And the free drug and moisture content are still low, and the biological activity and binding activity are still within the range of 100±30%. Additionally, the product has a good physical and chemical stability and light stability.

### Examples

The technical solution of the present invention will be further described below through specific embodiments. It should be noted that the embodiments are only exemplary, and not a limitation of the protection scope of the present invention. The invention may have other embodiments or can be practiced or carried out in a variety of ways.

### Laboratory Equipments Consumables and Reagents

### Laboratory Equipments

| Equipment name | Equipment manufacturer | Equipment model |
|---|---|---|
| pH meter | METTLER TOLEDO | SevenCompact S210 |
| pH meter | METTLER TOLEDO | Seven 8 |
| Analytical balance | Sartorius | SQP |
| | | Specifications: QUINTIX6102-1CN, |
| | | QUINTIX612-1CN, QUINTIX612-1CN |
| Analytical balance | METTLER TOLEDO | BSA623S |
| Analytical balance | METTLER TOLEDO | MS304TS |
| Peristaltic pump | LONGER | BT300-2J |
| Peristaltic pump | LONGER | BT300N |
| Filling pump | Flexicon | PF7 |
| Pulsating vacuum sterilizer | Shinva Medical | XG1.DTX-0.36 |
| | Instrument Co., Ltd. | |
| Biosafety cabinet | SUZHOU ANTAI AIR | BSC-130011A2 |
| | TECH CO., LTD. | |
| Microfluidic imager | Protein Simple | MFI5200 |
| Osmometer | Gonotec | OSMOMAT 3000 |
| Constant temperature and | memmert | HPP400 |
| humidity box | | |
| High temperature dry oven | Thermo SCIENTIFIC | Heratherm OMH100-S |
| Ultra low temperature | Thermo | 88300V |
| refrigerator | | |
| Freeze dryer | Telstar | LYOBETA 3PS |
| Lyophilizer | Christ | Epsilon 2-4 LSCplus |
| Differential Scanning | METTLER TOLEDO | DSC3 |
| Calorimeter | | |
| Constant temperature and | MMM | Climacell 222 |
| humidity light incubator | | |
| Density Viscometer | Anton Paar | DMA4100M&Lovis2000M |
| Magnetic stirrer | IKA | IKA ®RET basic |
| Trace moisture analyzer | Arizona Instrument LLC | Computrac Vapor Pro ® XL |
| Freeze drying microscope | Linkam | FDCS196 |
| system | | |

### Experimental Consumables

| Serial | | | | | |
|---|---|---|---|---|---|
| number | Name | Specification | | Manufacturer | Item No. |
| 3 | polycarbonate bottles | 5 | ml | Thermo Fisher | 3500-05 |
| 5 | Neutral borosilicate glass tube | 20 | ml | SCHOTT Pharmaceutical | Medium |
| | injection vial | | | Packaging Co., Ltd | Boron 2019 |
| 7 | Chlorinated butyl rubber stopper | Φ20 | mm | West | 1970-0033 |
| | with PTFE film coated for freeze- | | | | |
| | drying | | | | |
| 8 | Aluminum-plastic combination cap | Φ20 | mm | Jiangsu Changjiang Lid | NA |

| | |
|---|---|
| for antibiotic bottle | Industry Co., Ltd. |

### Experimental Reagents

| Name | Manufacturer | Grade | Item No. |
|---|---|---|---|
| Sodium succinate | Merck | Medicinal grade | 1006815000 |
| Sodium hydroxide | Merck | Medicinal grade | 1370201000 |
| Sucrose | Pfanstiehl | Medicinal grade | S-124-2-MC |
| Polysorbate 20 | J. T. Baker | Medicinal grade | 4116-04 |

### 1. Stock Solution Stirring Experiment

### 1.1 The Purpose of the Experiment

Investigate the effect of stirring at room temperature on the quality of ADC.

### 1.2 Experimental Materials

Protein: C19388-20190719

### 1.3 Experimental Operation

(1) Divided the stock solution sample for stirring experiment into 4 vials, 10 ml/vial, concentration 20 mg/ml;
(2) The test samples and mixing operation are shown in the following table:

| Sample | | |
|---|---|---|
| serial | | |
| number | Processing method | Remarks |
| 1 | Let stand at room temperature, took and tested | Control |
| | samples after 60 minutes | |
| 2 | Stirred at room temperature until the liquid surface | Sample |
| | just saw the vortex for 15 minutes | |
| 3 | Stirred at room temperature until the liquid surface | Sample |
| | just saw the vortex for 30 minutes | |
| 4 | Stirred at room temperature until the liquid surface | Sample |
| | just saw the vortex for 60 minutes | |

(3) Testing items:

| | Let stand | | | |
|---|---|---|---|---|
| | at room | Stirred for | Stirred for | Stirred for |
| Testing items | temperature | 15 min | 30 min | 60 min |
| Protein concentration | ● | ● | ● | ● |
| SEC-HPLC | ● | ● | ● | ● |
| Sub-visible Particles | ● | ● | ● | ● |
| (MFI) | | | | |
| Charge heterogeneity | ● | ● | ● | ● |
| (CEX-HPLC) | | | | |
| CE-SDS | ● | ● | ● | ● |
| HIC-HPLC | ● | ● | ● | ● |
| Binding activity | ● | ● | ● | ● |
| Biological activity | ● | ● | ● | ● |

### 1.4 Test Results and Analysis

According to the test results (Table 1-1), compared with the sample standing at room temperature, the protein concentration, the purity of SEC-HPLC, the purity of nrCE-SDS, the purity of rCE-SDS, the proportion of the main peak of CEX-HPLC, HIC-HPLC (DAR value), biological activity and binding activity of the stock solution after stirring for 15 min, 30 min and 60 min had no significant changes, and the sub-visible particles decreased slightly.

TABLE 1-1
The results of the stock solution stirring test

| | Protein | | nrCE- | | | CEX-HPLC (%) | | | Binding | Biological | | |
|---|---|---|---|---|---|---|---|---|---|---|---|---|
| | concentration | SEC- | SDS | rCE- | HIC- | Acid | Main | Basic | activity | activity | MFI = ml | |
| Batch | (mg/ml) | HPLC(%) | (%) | SDS(%) | HPLC | peak | peak | peak | (%) | (%) | ≥10 μm | ≥25 μm |
| C193SS- | 20.5 | 99.0 | 96.4 | 97.3 | 1.79 | 29.9 | 57.0 | 13.1 | 95 | 101 | 126.21 | 8.20 |
| 20190719 | | | | | | | | | | | | |
| (Let stand at | | | | | | | | | | | | |
| room | | | | | | | | | | | | |
| temperature) | | | | | | | | | | | | |
| C19388- | 20.6 | 99.0 | 96.4 | 97.4 | 1.79 | 29.7 | 57.4 | 12.9 | 90 | 94 | 52.52 | 4.92 |
| 20190719 | | | | | | | | | | | | |
| {Stirred for 15 | | | | | | | | | | | | |
| min} | | | | | | | | | | | | |
| C19388- | 21.0 | 99.0 | 96.3 | 97.3 | 1.79 | 29.8 | 57.3 | 13.0 | 88 | 103 | 52.45 | 4.92 |
| 20190719 | | | | | | | | | | | | |
| {Stirred for 30 | | | | | | | | | | | | |
| min} | | | | | | | | | | | | |
| C19388- | 20.6 | 99.1 | 96.3 | 97.3 | 1.79 | 30.0 | 57.0 | 13.05 | 104 | 97 | 26.22 | 8.19 |
| 20190719 | | | | | | | | | | | | |
| {Stirred for 60 | | | | | | | | | | | | |
| min} | | | | | | | | | | | | |

### 1.5 Conclusion

In summary, the protein concentration, the purity of SEC-HPLC, the purity of nrCE-SDS, the purity of rCE-SDS, the proportion of acid peak, main peak and basic peak of CEX-HPLC, DAR value, sub-visible particles, biological activity and binding activity of C19388 stock solution after stirring for 60 minutes were relatively stable. Therefore, continuous stirring for 60 minutes under the condition that the vortex was just visible on the liquid surface had no effect on the quality of the C19388 stock solution.

### 2 Freeze-Dried Small-Scale Test

### 2.1 The Purpose of the Experiment

The ADC freeze-dried samples were prepared by the platform freeze-drying process, and the appearance after freeze-drying and the stability of ADC were investigated.

### 2.2 Experimental Materials

Protein: C19388-20190719

### 2.3 Experimental Operation

### 2.3.1 Solution Preparation

| Solution | |
|---|---|
| name | Solution components |
| C19388 | 10 mmol/L sodium succinate, 6% sucrose |
| formulation | (W/V), 0.02% polysorbate 20 (W/V), and |
| buffer | the pH is adjusted to 5.0 with sodium hydroxide. |

2.3.2 Experimental operation
(1) Took the protein stock solution C19388-20190719, thawed at room temperature, mixed well, took the appropriate amount of protein and formulation buffer, used a freeze-drying microscope and differential calorimetry scanner to determine the collapse temperature and glass state transition temperature of the protein stock solution and formulation buffer, respectively;
(2) Filling: In the clean bench, 21 vials of ADC stock solution, 5 ml/vial were filled, 6 vials were used for 0-hour detection after freeze-drying, and 12 vials were used for stability inspection at 40° C. after freeze-drying, 6 samples were sampled 2 weeks and 4 weeks for detection respectively; in addition, 48 vials formulation buffer solution were filled, 5 ml/vial. The freeze-drying parameters were as follows:

| | | | Freeze | |
|---|---|---|---|---|
| | | | drying | |
| | | Temperature | chamber | |
| Freeze-drying | | change rate | pressure | Time |
| steps | Temperature(° C.) | (° C./h) | (Pa) | (h) |
| Cooling of the | 20~−45 | 22 | Normal | 3 |
| pre-freezing | | | pressure | |
| Pre-freezing | −45 | 0 | Normal | 8 |
| | | | pressure | |
| Heating up | −45~−25 | 5 | 25 Pa | 4 |
| Primary drying | −25 | 0 | 25 Pa | 40 |
| Heating up | −25~25 | 10 | 25 Pa | 5 |
| Secondary drying | 25 | 0 | 25 Pa | 8 |

(3) Testing items and time points:

| Testing items | Before freeze-drying | 0-hour | 2 weeks | 4 weeks |
|---|---|---|---|---|
| Moisture | / | ● | ● | ● |
| Reconstitution time | / | ● | ● | ● |
| Appearance | / | ● | ● | ● |
| Osmolality | ● | ● | / | / |
| Sub-visible particles | / | ● | ● | ● |
| (MFI) | | | | |
| pH | / | ● | / | / |
| Protein concentration | / | ● | ● | ● |
| SEC-HPLC | / | ● | ● | ● |
| CEX-HPLC | / | ● | ● | ● |
| HIC-HPLC | / | ● | ● | ● |
| nrCE-SDS | / | ● | ● | ● |
| Binding activity | / | ● | ● | ● |
| Biological activity | / | ● | ● | ● |

### 2.4 Test Results and Analysis

### 2.4.1 Test Results of Glass State Transition Temperature/Collapse Temperature

According to the Tg′/Tc test results (Table 2-1): The Tg′/Tc values of the stock solution samples were all higher than the Tg′/Tc values of the formulation buffer.

TABLE 2-1
Test results of freeze-dried small-scale test-Tg′/Tc

| | Glass state | |
|---|---|---|
| | transition | Collapse |
| | temperature | temperature |
| sample name | (° C.) | (° C.) |
| Formulation buffer | −32.4 | −32.8 |
| C19388 stock solution | −28.4 | −24.2 |

### 2.4.2 Stability Test Results of Freeze-Dried Small-Scale Test Samples

According to the stability test results (Table 2-2), there was no significant difference in osmotic pressure of ADC samples before and after freeze-drying. There was no significant change in the appearance between the ADC samples at 0-hour and accelerated for 2 weeks and 4 weeks at 40° C., and the reconstitution time were all about 1 minute, which were all short, protein concentration, the purity of SEC-HPLC, the purity of nrCE-SDS, the proportion charge variants of CEX-HPLC, DAR value, sub-visible particles, biological activity and binding activity were all not significantly different, and the moisture content compared to 0 h increased slightly, but all were less than 2%, at a low level.

TABLE 2-2
The stability test results of freeze-dried small-scale test sample

| | | | Time point | | | | | | |
|---|---|---|---|---|---|---|---|---|---|
| Sample batch | | | Before freeze- | | | | | | |
| number | Testing items | | drying | 0-hour | | 2 weeks | | 4 weeks | |
| C19388- | Osmolality | | 216/218/219 | 211/213/212 | | / | | / | |
| 20190719 | (mOsmol/kg) | | | | | | | | |
| | Reconstitution | | / | 58/66 | | 39/40 | | 47/56 | |
| | time (s) | | | | | | | | |
| | Appearance | | / | White, loose soild, | | White, loose | | White, loose | |
| | | | | the walls around | | soild, the walls | | soild, the walls | |
| | | | | were detached | | around were | | around were | |
| | | | | | | detached | | detached | |
| | Moisture (%) | | / | 0.92/0.85/0.73 | | 1.14/1.32/1.12 | | 1.55/1.40/1.43 | |
| | pH | | / | 5.1 | | / | | / | |
| | Protein | | / | 20.2 | | 20.4 | | 20.7 | |
| | concentration | | | | | | | | |
| | (mg/ml) | | | | | | | | |
| | SEC-HPLC (%) | | / | 99.0 | | 99.0 | | 98.9 | |
| | CEX- | Acid | / | 30.0 | | 30.9 | | 31.4 | |
| | HPLC (%) | peak | | | | | | | |
| | | Main | / | 57.0 | | 56.6 | | 55.9 | |
| | | peak | | | | | | | |
| | | Basic | / | 13.0 | | 12.5 | | 12.7 | |
| | | peak | | | | | | | |
| | HIC-HPLC (DAR | | / | 1.78 | | 1.78 | | 1.78 | |
| | value) | | | | | | | | |
| | nrCE-SDS (%) | | / | 96.3 | | 96.0 | | 96.3 | |
| | Biological activity | | / | 99 | | 99 | | 97 | |
| | (%) | | | | | | | | |
| | Binding activity (%) | | / | 100 | | 91 | | 100 | |
| | MFI | ≥10 μm | / | 39.34 | 19.67 | 21.31 | 18.94 | 27.85 | 14.76 |
| | (#/ml) | ≥25 μm | | 1.64 | 1.64 | 2.37 | 4.74 | 3.28 | 3.28 |

### 2.5 Conclusion

After 4 weeks of acceleration at 40° C., the quality attributes of the freeze-dried small-scale ADC samples did not change significantly, that is, the quality attributes of the lyophilized products of C19388 remained stable when accelerated at 40° C. for 4 weeks.

### 3. Optimization of Freeze-Drying Process

### 3.1 The Purpose of the Experiment

According to the freeze-drying curve of the small-scale freeze-drying test, a 0.5 m² freeze-drying machine was used to optimize the pre-freezing, primary drying and secondary drying parameters.

### 3.2 Experimental Materials

Protein: C19388-20190716

### 3.3 Experimental Operation

### 3.3.1 Solution Preparation

| Solution name | Solution components |
|---|---|
| C19388 formulation | 10 mmol/L sodium succinate, 6% sucrose (W/V), |
| buffer | 0.02% polysorbate 20 (W/V), and the |
| | pH is adjusted to 5.0 with sodium hydroxide. |

### 3.3.2 Specific Experimental Operation

In the process of freeze-drying optimization, a layer was used for freeze-drying optimization, and the specific steps were as follows:
(1) Pre-freezing optimization: according to the freeze-dried small-scale test data and Tg′/Tc test results, the pre-freezing temperature was selected to be −45° C. to optimize the cooling rate in the pre-freezing stage. Filled 116 vials of formulation buffer, 5 ml/vial. Before freeze-drying, took 35 vials in the middle area of the plate layer for marking and weighing for sublimation rate test. Tested the sublimation rate according to the parameters in the following table. After the test was completed, pressed the plug and took out of the tank, measured the quality of each marked sample, calculated the quality difference before and after freeze-drying, that is, the amount of water sublimation, and calculated the sublimation rate. The freeze-drying parameters were as follows:

| | Freeze drying parameters | | | | | | | |
|---|---|---|---|---|---|---|---|---|
| | Pre-freezing | | | | Primary drying | | | |
| | Cooling | Plate layer | Holding | Vacuum | Heating | Plate layer | Holding | Vacuum |
| Serial | rate | temperature | time | degree | rate | temperature | time | degree |
| number | (° C./min) | (° C.) | (h) | (Pa) | (° C./h) | (° C.) | (h) | (Pa) |
| 1 | 0.3 | −45° C. | 4 | Non- | 5 | 25 | 15 | 25 |
| 2 | 1.0 | | | vacuum | | | | |

(2) Primary drying optimization: Pre-freeze using selected pre-freezing parameters to optimize primary drying plate layer temperature and freeze-drying chamber pressure. Filled 116 vials of formulation buffer, 5 ml/vial. Before freeze-drying, took 35 vials in the middle area of the plate layer for marking and weighing for sublimation rate test. Test the sublimation rate according to the parameters in the following table. After the test was completed, pressed the plug and took out of the tank, measured the quality of each marked sample, calculated the quality difference before and after freeze-drying, that is, the amount of water sublimation, and calculated the sublimation rate. The freeze-drying parameters were as follows:

| | Freeze drying parameters | | | | | | | |
|---|---|---|---|---|---|---|---|---|
| | Pre-freezing | | | | Primary drying | | | |
| | Cooling | Plate layer | Holding | Vacuum | Plate layer | Vacuum | Heating | Holding |
| Serial | rate | temperature | time | degree | temperature | degree | rate | time |
| number | (° C./min) | (° C.) | (h) | (Pa) | (° C./h) | (Pa) | (° C./h) | (h) |
| 1 | 1 | −45° C. | 4 | Non- | −25 | 25 | 5 | 15 |
| 2 | | | | vacuum | −20 | 25 | 6 | |
| 3 | | | | | −20 | 35 | 6 | |
| 4 | | | | | −25 | 35 | 5 | |

After the sublimation rate test was completed, selected the better parameters for a complete primary drying process test, filled 116 vials of formulation buffer, 5 ml/vial, and checked the appearance after the freeze-drying. The freeze-drying parameters were as follows:

| | Freeze drying parameters | | | | | | | |
|---|---|---|---|---|---|---|---|---|
| | Pre-freezing | | | | Primary drying | | | |
| | Cooling | Plate layer | Holding | Vacuum | Plate layer | Vacuum | Heating | Holding |
| Serial | rate | temperature | time | degree | temperature | degree | rate | time |
| number | (° C./min) | (° C.) | (h) | (Pa) | (° C./h) | (Pa) | (° C./h) | (h) |
| 1 | 1 | −45° C. | 4 | Non- | −20 | 35 | 6 | 43 |
| 2 | | | | vacuum | −20 | 25 | | |

(3) Secondary drying optimization: using pre-freezing and primary drying optimization parameters to optimize the temperature and holding time of the secondary drying plate layer. Filled 116 vials of formulation buffer solution, 5 ml/vial, freeze-dried with the parameters in the following table, after the secondary drying pressure and temperature rise test completed, pressed the plug in vacuum, took out of the tank, and detected the appearance and moisture. The freeze-drying parameters were as follows:

| | Freeze drying parameters | | | | | | | | | | | |
|---|---|---|---|---|---|---|---|---|---|---|---|---|
| | Pre-freezing | | | | Primary drying | | | | Secondary drying | | | |
| | Cooling | Plate layer | Holding | Vacuum | Plate layer | Vacuum | Heating | Holding | Plate layer | Vacuum | Heating | Holding |
| Serial | rate | temperature | time | degree | temperature | degree | rate | time | temperature | degree | rate | time |
| number | (° C./min) | (° C.) | (h) | (Pa) | (° C./h) | (Pa) | (° C./h) | (h) | (° C./h) | (Pa) | (° C./min) | (h) |
| 1 | 1 | −45° C. | 4 | Non- | −20 | 25 | 6 | 43 | 30 | 25 | 0.3 | 8 |
| | | | | vacuum | | | | | | | | |

(4) Confirmation of optimization of freeze-drying: Filled 10 samples, 5 ml/vial, 4 vials for temperature probe, 6 vials for 0 h detection, and filled 106 vials of formulation buffer solution, 5 ml/vial. Freeze-dry was carried out according to the optimized parameters of pre-freezing, primary drying and secondary drying. The freeze-drying parameters were as follows:

| | Freeze drying parameters | | | |
|---|---|---|---|---|
| | | | Freeze | |
| | | | drying | |
| | | Temperature | chamber | |
| | Temperature | change rate | pressure | Time |
| Freeze drying stage | (° C.) | (° C./h) | (Pa) | (h) |
| Cooling of the pre- | 20~−45 | 65 | Normal | 1 |
| freezing | | | pressure | |
| Pre-freezing | −45 | 0 | Normal | 4 |
| | | | pressure | |
| Heating up | −45~−20 | 6 | 25 | 4 |
| Primary drying | −20 | 0 | 25 | 43 |
| Heating up | −20~30 | 17 | 25 | 3 |
| Secondary drying | 30 | 0 | 25 | 8 |

Test items: appearance, moisture, reconstitution time, osmotic pressure, sub-visible particles (MFI), pH, protein concentration, SEC-HPLC, binding activity, biological activity.

### 3.4 Test Results and Analysis

### 3.4.1 The Results of the Pre-Freezing Optimization

According to the freeze-dried small-scale test data and the Tg′/Tc test results, the pre-freezing temperature was selected as −45° C., and the cooling rate in the pre-freezing stage was optimized. According to the results (Table 3-1), the statistical analysis showed that the sublimation rate was significantly higher than 0.3° C./min (P<0.05) when the pre-freezing cooling rate was 1° C./min, so the pre-freeze cooling rate of 1° C./min was selected for subsequent optimization experiments.

TABLE 3-1
Freeze-drying process optimization- The result of the pre-freezing optimization

| | | | | Average | |
|---|---|---|---|---|---|
| | | Freeze drying parameters | | sublimation | |
| Serial | | Cooling rate | | rate | |
| number | Phase | (° C./min) | Other parameters | (g/h/vial) | RSD (%) |
| 1 | Pre-freezing | 0.3 | Pre-freezing: plate layer | 0.178 | 6.20 |
| 2 | | 1.0 | temperature −45° C., | 0.184 | 4.94 |
| | | | maintained for 4 h | | |
| | | | Primary drying: plate layer | | |
| | | | temperature −25° C., heating | | |
| | | | time 4 h, maintained for 15 h | | |

### 3.4.2 The Results of Primary Drying Optimization

The results (Table 3-2 and Table 3-3) showed that when the chamber pressure was 35 Pa, the appearance of the freeze-dried formulation buffer sample collapsed, so the primary drying plate layer temperature was −20° C., and the chamber pressure was 25 Pa for the subsequent second drying optimization.

TABLE 3-2
Freeze-drying process optimization- sublimation rate of primary drying

| | | Freeze drying parameters | | | Average | |
|---|---|---|---|---|---|---|
| | | Plate layer | Chamber | | sublimation | |
| Serial | | temperature | pressure | | rate | RSD |
| number | Phase | (° C.) | (Pa) | Other parameters | (g/h/vial) | (%) |
| 1 | Primary | −25 | 25 | Pre-freezing: plate layer | 0.191 | 6.15 |
| 2 | drying | −20 | 25 | temperature −45° C., cooling rate | 0.252 | 6.53 |
| 3 | | −20 | 35 | 1° C./min, maintained for 4 h | 0.262 | 5.34 |
| 4 | | −25 | 35 | Primary drying: heating time 4 h, | 0.193 | 6.11 |
| | | | | maintained for 15 h | | |

TABLE 3-3
Freeze-drying process optimization- complete primary drying test

| | Freeze drying parameters | | | |
|---|---|---|---|---|
| | Plate layer | Chamber | | |
| | temperature | pressure | | Appearance after |
| Phase | (° C.) | (Pa) | Other parameters | primary drying |
| Primary | −20 | 35 | Pre-freezing: plate layer | The walls around were |
| drying | | | temperature −45° C., cooling rate | detached and powder |
| | | | 1° C./min, maintained for 4 h | cake collapsed |
| | −20 | 25 | Primary drying: heating time 4 h, | The walls around were |
| | | | maintained for 43 h | detached and the |
| | | | | bottom was slightly |
| | | | | shrunk |

Note:
completed the visual inspection within 10 minutes after the sample was out of the tank

### 3.4.3 The Results of Secondary Drying

The results (Table 3-4) showed that after freeze-drying, there were no significant difference in appearance and moisture content between freeze-dried formulation buffer samples and between freeze-dried protein samples, the appearance was good, and the moisture content were low.

TABLE 3-4
Freeze-drying process optimization- The results of the pre-freezing optimization

| | Freeze drying parameters | | | | | | |
|---|---|---|---|---|---|---|---|
| | Plate layer | Holding | | | | | |
| | temperature | time | | Appearance | | Moisture (%) | |
| Phase | (° C.) | (h) | Other parameters | Buffer | Samples | Buffer | Samples |
| Secondary | 30 | 8 | Pre-freezing: plate layer | The powder | The powder | 1.48 | 0.77 |
| drying | | | temperature −45° C., cooling | cake was | cake was | 1.26 | 0.84 |
| | | | rate 1° C./min, maintained | intact, the | intact, the | 1.26 | 0.88 |
| | | | for 4 h; | walls | walls | | |
| | | | Primary drying: plate layer | around were | around were | | |
| | | | temperature −20° C., heating | detached | detached | | |
| | | | time 4 h, maintained for 43 h | and the | | | |
| | | | Secondary drying: heating | bottom was | | | |
| | | | time 3 h | shrunk | | | |

Note:
completed the visual inspection within 10 minutes after the sample was out of the tank

### 3.4.4 The Test Results of Freeze-Drying Optimization Confirmation

According to the test results (Table 3-5) of the product using the above optimized parameters: the appearance of the protein samples was good at 0-hour, the reconstitution time was within 1 minute, the moisture content and particle level were both low, and the protein concentration and osmolality after reconstitution had no significant changes compared to the stock solution. The purity of SEC-HPLC did not decrease significantly, and the binding activity and biological activity were both high.

TABLE 3-5
Freeze-drying process optimization- the results
of the freeze-drying optimization confirmation

| | | | Time point | |
|---|---|---|---|---|
| Batch | Testing items | | 0-hour | |
| C19388-20190827 | Moisture (%) | | 0.71/0.89/0.85 | |
| | Reconstitution time (s) | | 50/39 | |
| | Appearance | | The powder cake was intact, | |
| | | | the walls around were detached | |
| | Osmolality (mOsmol/kg) | | 225/220/219 | |
| | pH | | 5.1 | |
| | Protein concentration (mg/ml) | | 20.4 | |
| | SEC-HPLC (%) | | 99.1 | |
| | Binding activity(%) | | 99 | |
| | Biological activity(%) | | 103 | |
| | Sub-visible particles | ≥10 μm | 204.81 | 18.02 |
| | (#/ml) | ≥25 μm | 60.67 | 1.64 |

### 3.5 Conclusion

In summary, the optimized parameters of freeze-drying were as follows:

| | Freeze drying parameters | | | |
|---|---|---|---|---|
| | | | Freeze | |
| | | | drying | |
| | | Temperature | chamber | |
| | Temper- | change rate | pressure | Time |
| Freeze drying stage | ature(° C.) | (° C./h) | (Pa) | (h) |
| Cooling of the pre- | 20~−45 | 65 | Normal | 1 |
| freezing | | | pressure | |
| Pre-freezing | −45 | NA | Normal | 4 |
| | | | pressure | |
| Heating up | −45~−20 | 6 | 25 | 4 |
| Primary drying | −20 | NA | 25 | 43 |
| Heating up | −20~30 | 17 | 25 | 3 |
| Secondary drying | 30 | NA | 25 | 8 |

### 4. The Confirmation of Full-Tank Freeze-Drying Process

### 4.1 The Purpose of the Experiment

The developed freeze-drying process was scaled up for full-tank, and the freeze-drying parameters for the full-tank were determined.

### 4.2 Experimental Materials

Formulation buffer which is the same as example 2.3.1 and example 3.3.1.

### 4.3 Experimental Steps

(1) Filled 360 vials of formulation buffer solution with the adjusted filling volume, 5.3 ml/vial;
(2) Used experiment 3 freeze-drying optimized parameters, and appropriately adjusted the drying and holding time, and freeze-dried formulation buffer with a 0.5 m² scale to determine the final full-tank freeze-drying parameters. The parameters were set as follows:

| | | | Freeze | |
|---|---|---|---|---|
| | | | drying | |
| | | Temperature | chamber | |
| | Temper- | change rate | pressure | Time |
| Freeze drying stage | ature(° C.) | (° C./min) | (Pa) | (h) |
| Cooling of the pre- | 20~−45 | 1.0 | Normal | 1 |
| freezing | | | pressure | |
| Pre-freezing | −45 | NA | Normal | 4 |
| temperature | | | pressure | |
| Primary drying | −45~−20 | 0.42 | 25 | 1 |
| Primary drying | −20 | NA | 25 | 43 |
| temperature | | | | |
| Primary drying | −20 | NA | 25 | 2 |
| temperature | | | | |
| Primary drying | −20 | NA | 25 | 2 |
| temperature | | | | |
| Primary drying | −20 | NA | 25 | 2 |
| temperature | | | | |
| Secondary drying | −20~30 | 0.42 | 25 | 2 |
| Secondary drying | 30 | NA | 25 | 8 |
| temperature | | | | |

(3) After the freeze-drying was completed, the appearance and moisture of the freeze-dried formulation buffer were measured.

### 4.4 Results Analysis and Conclusion

The results (Table 4-1) showed that there was no significant difference in appearance of all freeze-dried formulation buffer samples after freeze-drying, and the moisture content was low.

TABLE 4-1
The results of full-tank freeze-drying process confirmation

| sample name | Appearance | Moisture (%) |
|---|---|---|
| Formulation buffer | The walls around were | 1.23/1.40/1.42/ |
| | detached and the bottom was | 1.44/1.19/1.16/1.49 |
| | shrunk | |

In summary, the freeze-drying parameter can be used for sample full-tank preparation.

### 5. Process Development Confirmation of Batch Preparation and Stability Research

### 5.1 The Purpose of the Experiment

The preparation process development confirmed for batch freeze-dried drug products, and examined the stability.

### 5.2 Experimental Materials

Protein: C19388 stock solution, batch number: C19388-20190909.
Protein: C19388 stock solution, batch number: GQ1001-190902.

### 5.3 Experimental Steps of Batch Number: C19388-20190909

### 5.3.1 Solution Preparation

| Solution name | Solution components |
|---|---|
| C19388 formulation | 10 mmol/L sodium succinate, 6% sucrose (W/V), |
| buffer | 0.02% polysorbate 20 (W/V), and the |
| | pH is adjusted to 5.0 with sodium hydroxide. |

5.3.2 Experimental operation
(1) Filled 183 samples, 5.3 ml/vial, 9 of which were used to place temperature probes, and the rest were filled with formulation buffer to the full-tank;
(2) The parameters were determined by the experiment 4, freeze-dried in a full-tank at the scale of 0.5 m². The freeze-drying parameters were as follows:

| | | | Freeze | |
|---|---|---|---|---|
| | | | drying | |
| | | Temperature | chamber | |
| | Temperature | change rate | pressure | Time |
| Freeze drying stage | (° C.) | (° C./min) | (Pa) | (h) |
| Cooling of the pre- | 20~−45 | 1.0 | Normal | 1 |
| freezing | | | pressure | |
| Pre-freezing | −45 | NA | Normal | 4 |
| temperature | | | pressure | |
| Primary drying | −45~−20 | 0.42 | 25 | 1 |
| Primary drying | −20 | NA | 25 | 49 |
| temperature | | | | |
| Secondary drying | −20~30 | 0.42 | 25 | 2 |
| Secondary drying | 30 | NA | 25 | 8 |
| temperature | | | | |

(3) Put the drug product of C19388 at 40° C. for acceleration, sampled and tested at 0-hour, 2 weeks and 4 weeks;
(4) Testing items:

| | Time point | | |
|---|---|---|---|
| Testing items | 0-hour | 2 weeks | 4 weeks |
| Appearance/color after reconstitution | ● | ● | ● |
| Reconstitution time | ● | ● | ● |
| Visible particle | ● | ● | ● |
| Protein concentration | ● | ● | ● |
| pH | ● | ● | ● |
| Osmotic pressure | ● | ● | ● |
| Clarity | ● | ● | ● |
| Insoluble particles | ● | ● | ● |
| Moisture | ● | ● | ● |
| The purity of SEC-HPLC | ● | ● | ● |
| The purity of CE-SDS | ● | ● | ● |
| Charge heterogeneity | ● | ● | ● |
| HIC-HPLC | ● | ● | ● |
| Free drug | ● | ● | ● |
| Binding activity | ● | ● | ● |
| Biological activity | ● | ● | ● |

### 5.3.3 Test Results and Analysis

According to the results of the stress temperature stability inspection of the confirmed batch of drug products (Table 5-1), compared with the results at 0-hour, the sample appearance/color, pH, osmolality, clarity, protein concentration, and sub-visible particles, DAR value did not change significantly when placed at 40° C. for 4 weeks, the reconstitution time was still within 1 minute, and one visible particle was detected in samples at 0-hour and 4 weeks respectively. At 4 weeks, the purity of SEC-HPLC, the purity of CE-SDS, the charge variants of CEX-HPLC did not change significantly, the free drug and moisture content did not increase significantly, and the binding activity and biological activity remained both stable.

In addition, only two of the samples at 0-hour and 4 weeks showed one visible particle. At 2 weeks of acceleration, there was no visible particle in all samples, and the level of sub-visible particle in the samples was low at 0-hour, 2 weeks, and 4 weeks. Therefore, the visible particles detected should be caused by the insufficiently clean laboratory environment.

TABLE 5-1
The results of process development confirmation batch drug product stability test

| | | Batch | | |
|---|---|---|---|---|
| | | C19388- | C19388-20190909 (2 | C19388-20190909 (4 |
| Testing items | | 20190909(0-hour) | weeks at 40° C.) | weeks at 40° C.) |
| Appearance/color after | | Light yellow liquid, lighter | Light yellow liquid, | Light yellow liquid, |
| reconstitution | | than yellow No. 2 standard | lighter than yellow | lighter than yellow No. |
| | | colorimetric solution (ChP) | No. 2 standard | 2 standard colorimetric |
| | | Light yellow liquid, | colorimetric solution | solution (ChP) |
| | | lighter than Y5 (Ph. Eur) | (ChP) | Light yellow liquid, |
| | | | Light yellow liquid, | lighter than Y5 |
| | | | lighter than Y5 | (Ph. Eur) |
| | | | (Ph. Eur) | |
| Reconstitution time (s) | | 32/37/39 | 32/43/48 | 37/38/44 |
| Visible particles | | One white fibrous particle | No obvious | One gray block |
| | | smaller than 2 mm was | visible | smaller than 2 mm |
| | | detected in one of | particle | was detected in one |
| | | four samples | | of four samples |
| Protein concentration | | 20.2 | 20.6 | 21.3 |
| (mg/ml) | | | | |
| pH | | 5.1 | 5.1 | 5.1 |
| Osmolality | | 223 | 219 | 221 |
| (mOsmol/kg) | | | | |
| Clarity (NTU) | | 2.5 | 2.5 | 2.7 |
| Sub-visible | ≥10 μm | 43 | 30 | 70 |
| particles (#/ml) | ≥25 μm | 3 | 5 | 18 |
| Moisture (%) | | 0.82 | 0.87 | 0.90 |
| SEC-HPLC (%) | | 99.2 | 98.9 | 98.9 |
| nrCE-SDS (%) | | 96.3 | 96.3 | 96.4 |
| rCE-SDS (%) | | 97.2 | 97.3 | 97.5 |
| CEX-HPLC | Acid | 31.2 | 31.6 | 31.6 |
| (%) | peak | | | |
| | Main | 57.3 | 55.9 | 55.8 |
| | peak | | | |
| | Basic | 11.5 | 12.5 | 12.6 |
| | peak | | | |

| | | | |
|---|---|---|---|
| HIC-HPLC (DAR | 1.79 | 1.79 | 1.78 |
| value) | | | |
| Free Drug (%) | <0.005 | <0.005 | <0.005 |
| Binding activity (%) | 103 | 94 | 96 |
| Biological activity (%) | 105 | 108 | 101 |

### 5.3.4 Conclusion

In summary, after 4 weeks of stability inspection at 40° C., all indicators of the drug product of C19388 remained stable, that is, the freeze-dried drug product of C19388 had good stability.

### 5.3.5 Experimental Steps of Batch Number: GQ1001-190902

The preparation process development further confirmed by examining the stability of another batch product. The stability of the product of batch number GQ1001-190902 prepared by the present invention was determined at 40° C. using the same buffer solution and the same method as the product of batch number C19388-20190909. The products were sampled and detected at 0-hour, 2 weeks, 4 weeks, 8 weeks and 12 weeks. The testing items and the results are as following table:

TABLE 5-1′
The results of process development confirmation batch drug product stability test (Batch: GQ1001-190902)

| Testing items | | T0 | 2 W | 4 W | 8 W | 12 W |
|---|---|---|---|---|---|---|
| Charge variant | Main peak | 58.7% | 58.5% | 57.1% | 54.3% | 53.1% |
| (CEX-HPLC) | Acidic peak | 29.3% | 29.3% | 30.5% | 31.4% | 32.5% |
| | Basic peak | 12.0% | 12.2% | 12.4% | 14.3% | 14.4% |
| Purity(SEC- | Monomer | 99.1% | 99.0% | 98.9% | 98.9% | 98.8% |
| HPLC) | Aggregate | 0.9% | 1.0% | 1.1% | 1.1% | 1.2% |
| | Fragment | 0.0% | 0.0% | 0.0% | 0.0% | 0.0% |
| Purity(Non- | Monomer | 96.9% | 96.8% | 96.6% | 96.7% | 96.9% |
| reduced CE-SDS) | Fragment | 3.1% | 3.2% | 3.5% | 3.3% | 3.1% |
| Purity (Reduced | LC + HC | 96.8% | 96.8% | 96.9% | 96.9% | 97.0% |
| CE-SDS) | NGHC (non- | 0.9% | 0.8% | 0.8% | 0.8% | 0.8% |
| | glycosilation HC) | | | | | |
| Free drug | | <0.005% | <0.005% | <0.005% | <0.005% | <0.005% |
| Biological activity (Cell-Based Assay) | | 100% | 121% | 90% | 92% | 90% |
| Binding Activity (ELISA) | | 100% | 102% | 92% | 107% | 99% |
| Protein Content (UV) | | 101 mg/Vial | 101 mg/Vial | 104 mg/Vial | 103 mg/Vial | 101 mg/Vial |
| pH (pH Test) | | 5.0 | 5.1 | 5.0 | 5.1 | 5.0 |
| DAR (HIC-HPLC) | D0 | 0.7% | 0.7% | 0.7% | 0.7% | 0.8% |
| | DAR | 1.81 | 1.81 | 1.81 | 1.81 | 1.81 |

According to the results above Table 5-1′, compared with the results at 0-hour, the pH, protein content and DAR value of the samples did not change significantly when placed at 40° C. for 12 weeks. At 12 weeks, the purity of SEC-HPLC, the purity of CE-SDS (including non-reduced CE-SDS and reduced CE-SDS), the charge variants of CEX-HPLC did not change significantly. The free drug did not change, the binding activity remained stable, and the difference of the biological activity was also in the range of acceptance criteria (60%-140%). In summary, after 12 weeks of stability inspection at 40° C., all indicators of the drug product of GQ1001-190902 remained stable, that is, the freeze-dried drug product of GQ1001-190902 had good stability.

### 5.4 Further Confirmation of Accelerated Experiments and Stability Experiments

### 5.4.1 The Purpose of the Experiments

The experiment was used to further verify the above accelerated and long-term experiments.

### 5.4.2 Experimental Materials

Protein: C19388 stock solution, batch number: C19388-20190909.
Protein: C19388 stock solution, batch number: GQ1001-190902.

### 5.4.3 Experimental Operation

The experiment specific process was similar with example 2.3.2 and example 3.3.2. The results of the long-term experiment (5±3° C.) (Table 5-2) and accelerated experiment (25±2° C.) (Table 5-3) were as follows:

TABLE 5-2
Long-Term (5 ± 3° C.) Stability Data of GQ1001 Drug Product (Batch: C19388-20190909)

| Items | | 0 M | 1 M | 3 M | 6 M | 9 M | 12 M | 18 M | 24 M |
|---|---|---|---|---|---|---|---|---|---|
| Purity | monomer | 99.2% | 99.0% | 99.0% | 99.0% | 99.0% | 99.1% | 99.1% | 99.0% |
| (SEC- | aggregates | 0.8% | 1.0% | 1.0% | 1.0% | 1.0% | 1.0% | 0.9% | 1.0% |
| HPLC) | fragment | 0.0% | 0.0% | 0.0% | 0.0% | 0.0% | 0.0% | 0.0% | 0.0% |
| Purity | monomer | 96.3% | 96.4% | 96.6% | 96.6% | 96.6% | 96.7% | 96.1% | 96.1% |
| (Non- | fragment | 3.7% | 3.6% | 3.4% | 3.4% | 3.4% | 3.3% | 3.9% | 4.0% |
| reduced | | | | | | | | | |
| CE-SDS) | | | | | | | | | |
| Purity | LC + HC | 97.2% | 97.2% | 97.2% | 97.1% | 96.9% | 97.4% | 96.8% | 97.6% |
| (Reduced | NGHC | 0.8% | 0.8% | 0.9% | 0.8% | 0.8% | 0.8% | 0.8% | 0.8% |
| CE-SDS) | | | | | | | | | |
| Charge | Main peak | 57.3% | 59.0% | 58.0% | 58.2% | 58.2% | 58.2% | 59.0% | 57.5% |
| variant | Aacidic | 31.2% | 30.5% | 30.5% | 29.3% | 30.5% | 29.5% | 29.5% | 29.1% |
| (CEX- | components | | | | | | | | |
| HPLC) | Basic | 11.5% | 10.5% | 11.6% | 12.5% | 11.3% | 12.3% | 11.6% | 13.4% |
| | components | | | | | | | | |
| DAR | D0 | 0.6% | 0.8% | 0.6% | 1.0% | 0.7% | 0.7% | 1.8% | 1.6% |
| (HIC- | DAR value | 1.79 | 1.78 | 1.82 | 1.80 | 1.82 | 1.81 | 1.79 | 1.82 |
| HPLC) | | | | | | | | | |
| Free drug (P31-DM1 α | | <0.005% | <0.005% | <0.005% | <0.005% | NA | NA | <0.005% | <0.005% |
| isomers) | | | | | | | | | |
| Biological activity | | 105% | 97% | 100% | 98% | 94% | 99% | 110% | 105% |
| (Cell-Based Assay) | | | | | | | | | |
| Binding activity | | 103% | 100% | 101% | 103% | 92% | 89% | 108% | 111% |
| (ELISA) | | | | | | | | | |
| Protein content (UV) | | 101 | 107 | 102 | 100 | 103 | 99 | 100 | 103 |
| pH (pH Test) | | 5.1 | 5.1 | 5.1 | 5.1 | 5.1 | 5.1 | 5.1 | 5.1 |
| polysorbate 20 (FLD- | | 0.23 | 0.22 | 0.26 | 0.26 | NA | 0.24 | 0.19 | 0.20 |
| HPLC) (mg/mL) | | | | | | | | | |
| Clarity | | 2.5 NTU | 2.6 NTU | 2.6 NTU | 2.2 NTU | 2.1 NTU | 3.9 NTU | 2.5 NTU | 2.2 NTU |
| Sub- | ≥10 μm | 43/vial | 35/vial | 85/vial | 87/vial | 68/vial | 98/vial | 77/vial | 142/vial |
| visible | ≥25 μm | 3/vial | 0/vial | 7/vial | 7/vial | 5/vial | 5/vial | 3/vial | 7/vial |
| Particles | | | | | | | | | |
| (Light | | | | | | | | | |
| blockage | | | | | | | | | |
| method) | | | | | | | | | |

| | | | | | | | | |
|---|---|---|---|---|---|---|---|---|
| Osmolality | 223 | 224 | 222 | 220 | 221 | 218 | 213 | 218 |
| (mOsmol/kg) | | | | | | | | |
| Moisture content | 0.82% | 0.78% | 0.74% | 0.68% | 0.77% | 0.69% | 0.70% | 0.80 |

TABLE 5-3
The stability test (25 ± 2° C.) data of process development confirmation batch drug product

Testing items

| (analysis | Investigation time | | | |
|---|---|---|---|---|
| methods) | 0 M | 1 M | 3 M | 6 M |
| Charge | main peak: 57.3% | main peak: 56.4% | main peak: 57.0% | main peak: 56.8% |
| heterogeneity | acidic | acidic | acidic | acidic |
| (CEX-HPLC) | components: 31.2% | components: 31.0% | components: 31.9% | components: 30.1% |
| | basic | basic | basic | basic |
| | components: 11.5% | components: 12.6% | components: 12.2% | components: 13.1% |
| Purity (SEC- | monomer: 99.2% | monomer: 99.0% | monomer: 99.0% | monomer: 98.9% |
| HPLC) | aggregates: 0.8% | aggregates: 1.0% | aggregates: 1.1% | aggregates: 1.1% |
| | fragment: 0.0% | fragment: 0.0% | fragment: 0.0% | fragment: 0.0% |
| Purity (Non- | monomer: 96.3% | monomer: 96.3% | monomer: 96.5% | monomer: 96.5% |
| reduced CE- | fragment: 3.7% | fragment: 3.7% | fragment: 3.5% | fragment: 3.5% |
| SDS) | | | | |
| Purity | Sum of light and | Sum of light and | Sum of light and | Sum of light and |
| (Reduced CE- | heavy chain | heavy chain | heavy chain | heavy chain |
| SDS) | content: 97.2% | content: 97.4% | content: 97.1% | content: 97.2% |
| | non-glycosylated | non-glycosylated | non-glycosylated | non-glycosylated |
| | heavy chain: 0.8% | heavy chain: 0.8% | heavy chain: 0.9% | heavy chain: 0.8% |
| Free small | <0.005% | <0.005% | <0.005% | <0.005% |
| molecule drugs | | | | |
| (P31-DM1 α | | | | |
| isomer) | | | | |
| Biological | 105% | 102% | 105% | 106% |
| activity (Cell- | | | | |
| Based Assay) | | | | |
| Binding activity | 103% | 97% | 107% | 108% |
| (ELISA) | | | | |
| Protein content | 101 | 105 | 99 | 100 |
| (UV) | mg/vial | mg/vial | mg/vial | mg/vial |
| pH (pH test) | 5.1 | 5.1 | 5.1 | 5.1 |
| Clarity | 2.5 | 2.6 | 2.7 | 2.2 |
| (Turbidimeter | NTU | NTU | NTU | NTU |
| method) | | | | |
| Insoluble | ≥10 μm: 43/vial | ≥10 μm: 45/vial | ≥10 μm: 85/vial | ≥10 μm: 43/vial |
| particles (Light | | | | |
| blockage | ≥25 μm: 3/vial | ≥25 μm: 12/vial | ≥25 μm: 8/vial | ≥25 μm: 0/vial |
| method) | | | | |
| Osmolality | 223 | 222 | 221 | 219 |
| | mOsmol/kg | mOsmol/kg | mOsmol/kg | mOsmol/kg |
| Moisture content | 0.82% | 0.80% | 0.75% | 0.81% |
| (Coulometry) | | | | |
| DAR (HIC- | D0: 0.6% | D0: 0.8% | D0: 0.6% | D0: 1.0% |
| HPLC) | DAR: 1.79 | DAR: 1.78 | DAR: 1.82 | DAR: 1.80 |

### 5.4.4 Test Results and Analysis

According to the results of the long-term stability inspection of the confirmed batch of drug products (Table 5-2), compared with the results at 0 month the sample pH, osmolality, clarity, protein concentration, and DAR value did not change significantly when placed at 5° C. for 24 months. At 24 months, the purity of SEC-HPLC of the sample, the purity of CE-SDS, the proportion of the main peak of CEX-HPLC did not decrease significantly, the free drug and moisture content did not increase significantly, and the binding activity and biological activity remained both stable.

According to the results of the accelerated experiment (Table 5-3), the purity, pH, osmolality, clarity, protein concentration, and sub-visible particles, DAR value did not change significantly when placed at 25° C. for 6 months. At 6 months, the purity of SEC-HPLC, the purity of CE-SDS, the proportion of the main peak of CEX-HPLC did not decrease significantly, the free drug and moisture content did not increase significantly, and the binding activity and biological activity remained both stable.

### 5.4.5 Conclusion

In summary, the freeze-dried drug product of C19388 had good stability.

### 5.4.6 Experimental Steps of Batch Number: GQ1001-190902

The preparation process development further confirmed by examining the stability of another batch product. The stability of the product of batch number GQ1001-190902 prepared by the present invention was determined using the same buffer solution and the same method as the product of batch number C19388-20190909 in Experiment 5.4.3. The products were sampled and detected at 0 month, 1 month, 3 month, 6 month, 9 month, 12 month, 18 month, 24 month and 36 month in the long-term stability experiment, and the products were sampled and detected at 0 month, 1 month, 2 months, 3 months and 6 months in the accelerated stability experiment under 25±2° C. The results of the long-term experiment (5±3° C.) (Table 5-2′) and accelerated experiment (25±2° C.) (Table 5-3′) were as follows:

TABLE 5-2′
Long-Term (5 ± 3° C.) Stability Data of GQ1001 Drug Product (Batch: GQ1001-190902)

| Items | | 0 M | 1 M | 3 M | 6 M | 9 M |
|---|---|---|---|---|---|---|
| Purity (SEC- | Monomer | 99.1% | 99.1% | 99.0% | 99.0% | 99.0% |
| HPLC) | Aggregate | 0.9% | 0.9% | 1.0% | 1.0% | 1.0% |
| | Fragment | 0.0% | 0.0% | 0.0% | 0.0% | 0.0% |
| Purity (Non- | Monomer | 96.9% | 97.1% | 96.8% | 96.6% | 96.6% |
| reduced CE- | Fragment | 3.1% | 2.9% | 3.2% | 3.5% | 3.4% |
| SDS) | | | | | | |
| Purity | LC + HC | 96.8% | 97.0% | 96.9% | 97.1% | 97.0% |
| (Reduced | NGHC (non- | 0.9% | 0.8% | 0.8% | 0.8% | 0.8% |
| CE-SDS) | glycosilation | | | | | |
| | HC) | | | | | |
| Charge | Main Peak | 58.7% | 59.0% | 58.2% | 59.8% | 60.0% |
| variant | Acidic | 29.3% | 29.3% | 29.6% | 29.3% | 29.3% |
| (CEX- | Component | | | | | |
| HPLC) | Basic | 12.0% | 11.7% | 12.3% | 10.9% | 10.7% |
| | Component | | | | | |
| DAR (HIC- | D0 | 0.7% | 0.6% | 1.0% | 0.7% | 0.7% |
| HPLC) | DAR | 1.81 | 1.81 | 1.81 | 1.82 | 1.81 |
| Free drug | | <0.005% | <0.005% | <0.005% | <0.005% | <0.005% |
| Biological activity | | 100% | 95% | 103% | 103% | 93% |
| Binding activity | | 100% | 98% | 98% | 106% | 99% |
| Protein content (UV) | | 101 | 102 | 96 | 104 | 98 |
| pH (pH Test) | | 5.0 | 5.1 | 5.0 | 5.1 | 5.0 |
| Appearance | | White cake - | NA | NA | NA | NA |
| | | like loose | | | | |
| | | solid | | | | |
| | Items | | 12 M | 18 M | 24 M | 36 M |
| | Purity (SEC- | Monomer | 99.1% | 98.9% | 99.0% | 99.0% |
| | HPLC) | Aggregate | 0.9% | 1.1% | 1.0% | 1.0% |
| | | Fragment | 0.0% | 0.0% | 0.0% | 0.0% |
| | Purity (Non- | Monomer | 96.9% | 96.7% | 97.2% | 96.3% |
| | reduced CE- | Fragment | 3.1% | 3.3% | 2.8% | 3.7% |
| | SDS) | | | | | |
| | Purity | LC + HC | 96.9% | 97.3% | 97.5% | 96.8% |
| | (Reduced | NGHC (non- | 0.8% | 0.7% | 0.8% | 1.0% |
| | CE-SDS) | glycosilation | | | | |
| | | HC) | | | | |
| | Charge | Main Peak | 57.6% | 57.6% | 58.9% | 57.5% |
| | variant | Acidic | 30.1% | 30.3% | 29.7% | 29.7% |
| | (CEX- | Component | | | | |
| | HPLC) | Basic | 12.3% | 12.1% | 11.4% | 12.9% |
| | | Component | | | | |
| | DAR (HIC- | D0 | 0.7% | 0.5% | 0.5% | 0.8% |
| | HPLC) | DAR | 1.81 | 1.83 | 1.82 | 1.83 |

| | | | | |
|---|---|---|---|---|
| Free drug | <0.005% | <0.005% | <0.005% | <0.005% |
| Biological activity | 99% | 100% | 103% | 95% |
| Binding activity | 104% | 110% | 98% | 107% |
| Protein content (UV) | 103 | 98 | 104 | 97 |
| pH (pH Test) | 5.1 | 5.1 | 5.0 | 5.1 |
| Appearance | White cake - | NA | White pie- | White pie- |

| | | |
|---|---|---|
| like loose | like loose | like loose |
| solid | solid | solid |

TABLE 5-3′
The stability test (25 ± 2° C.) data of process development confirmation batch drug product (Batch: GQ1001-190902)

| Items | | 0 M | 1 M | 2 M | 3 M | 6 M |
|---|---|---|---|---|---|---|
| Purity (SEC-HPLC) | Monomer | 99.1% | 99.0% | 99.0% | 99.0% | 99.0% |
| | Aggregate | 0.9% | 1.0% | 1.0% | 1.0% | 1.0% |
| | Fragment | 0.0% | 0.0% | 0.0% | 0.0% | 0.0% |
| Purity (Non-reduced CE-SDS) | Monomer | 96.9% | 97.1% | 96.8% | 96.8% | 96.5% |
| | Fragment | 3.1% | 2.9% | 3.2% | 3.2% | 3.5% |
| Purity (Reduced CE-SDS) | LC + HC | 96.8% | 96.9% | 96.9% | 97.0% | 97.1% |
| | NGHC (non- | 0.9% | 0.8% | 0.8% | 0.8% | 0.8% |
| | glycosilation HC) | | | | | |
| Charge variant (CEX-HPLC) | Main Peak | 58.7% | 58.8% | 57.9% | 57.2% | 58.3% |
| | Acidic Components | 29.3% | 29.1% | 30.0% | 29.8% | 30.3% |
| | Basic Components | 12.0% | 12.1% | 12.1% | 12.9% | 11.4% |
| DAR (HIC-HPLC) | D0 | 0.7% | 0.6% | 0.7% | 0.7% | 0.7% |
| | DAR | 1.81 | 1.81 | 1.81 | 1.81 | 1.82 |

| | | | | | |
|---|---|---|---|---|---|
| Free drug | <0.005% | <0.005% | <0.005% | <0.005% | <0.005% |
| Biological activity (Cell-Based Assay) | 100% | 94% | 88% | 99% | 90% |
| Binding activity (ELISA) | 100% | 93% | 100% | 97% | 99% |
| Protein content (UV) | 101 | 109 | 102 | 100 | 95 |
| pH (pH Test) | 5.0 | 5.1 | 5.0 | 5.0 | 5.1 |

According to the results of the long-term stability inspection of the confirmed batch GQ1001-190902 of drug products (Table 5-2′), compared with the result at 0 month, the pH, protein content and DAR value had no significant changes, the biological activity and binding activity were within 100=10% over the 36 months. The purity of SEC-HPLC, the purity of CE-SDS, the proportion of main peak of CEX-HPLC and the free drug did not change significantly. The appearance of the products after 36 months was almost the same as that at 0 month. There only were 70/vial of sub-visible particles at 36 month, which is much lower than the acceptance criteria of ≤6000/vial.

According to the results of the accelerated experiment of the confirmed batch GQ1001-190902 of drug products above (Table 5-3′), compared with the result at 0 month, the pH, protein content and DAR value had no significant changes, the biological activity and binding activity were within 100±15% over the 6 months. The purity of SEC-HPLC, the purity of CE-SDS, the proportion of main peak of CEX-HPLC and the free drug did not change significantly.

In summary, the freeze-dried drug product of batch GQ1001-190902 has good stability.

### 5.5 Illumination Experiment

### 5.5.1 The Purpose of the Experiment

Investigated the effect of light on the freeze-dried drug product.

### 5.5.2 Experimental Materials

Protein: C19388 drug product, batch number: C19388-20190909.
Protein: C19388 drug product, batch number: GQ1001-190902.

### 5.5.3 Experimental Operation

(1) Illumination experiment: The stability was investigated under strong light conditions (5000±500 lx) at 25° C., and the experiment was carried out under strong light, normal packaging, and shading for 12 days. The total illuminance was not less than 1.2×10⁶ lx·H, sampling on the 5^{th}, 10^{th} and 12^{th} days for detection.
(2) Testing items:

| | Time point | | |
|---|---|---|---|
| Testing items | 5 days | 10 days | 12 days |
| Appearance/color after reconstitution | ● | ● | ● |
| Visible particles | ● | ● | ● |
| Protein concentration | ● | ● | ● |
| pH | ● | ● | ● |
| Moisture | ● | ● | ● |
| SEC-HPLC | ● | ● | ● |
| The purity of CE-SDS | ● | ● | ● |
| Charge heterogeneity | ● | ● | ● |
| HIC-HPLC | ● | ● | ● |
| Free drug | ● | ● | ● |
| Binding activity | ● | ● | ● |
| Biological activity | ● | ● | ● |

### 5.5.4 Result Analysis and Conclusion

According to the test results (Table 5-4): compared with the sample shading for 5 days, when the protein was placed in the light (5000±500 lx) for 5 days, the appearance, protein concentration, pH, and DAR value had no significant changes, and the biological activity and binding activity were within 100±30%, the purity of SEC-HPLC, the purity of CE-SDS, and the proportion of main peak of CEX-HPLC did not decrease significantly, and the free drug and moisture content did not increase significantly. Visible particles were detected in samples protected from light and shading at 5 days, but only two of all samples showed a visible particles, and there was no visible particle in all samples at 10 days and 12 days, therefore, the visible particle detected in the sample should be caused by the laboratory environment. After being placed for 10 days and 12 days, compared with 10 days and 12 days of shading, the protein samples were colorless and no visible particle after reconstitution. There were no significant changes in protein concentration, pH, and DAR values. The purity of SEC-HPLC, the purity of CE-SDS, the proportion of the main peak of CEX-HPLC did not decrease significantly, the free drug and moisture content were still low, and the biological activity and binding activity were still within the range of 100±30%, that is, the protein stability was good. Therefore, light for 12 days (5000±500 lx) had no effect on the quality of C19388 protein, and the light stability was good.

However, during long-term storage, dark storage is more conducive to reducing the impact of environmental factors on the quality of the drug product. Therefore, it is recommended to store the drug product in dark.

TABLE 5-4
The test results of illumination experiment

| | | | Illumination | | | Avoid light | | | Shading | | |
|---|---|---|---|---|---|---|---|---|---|---|---|
| Batch | Testing items | | 5 days | 10 days | 12 days | 5 days | 10 days | 12 days | 5 days | 10 days | 12 days |
| C19388- | Appearance/ | | Light | Light | Light | Light | Light | Light | Light | Light | Light |
| 20190909 | color after | | yellow | yellow | yellow | yellow | yellow | yellow | yellow | yellow | yellow |
| | reconstitution | | liquid, | liquid, | liquid, | liquid, | liquid, | liquid, | liquid, | liquid, | liquid, |
| | | | lighter | lighter | lighter | lighter | lighter | lighter | lighter | lighter | lighter |
| | | | than | than | than | than | than | than | than | than | than |
| | | | Y5(Ph. | Y5(Ph. | Y5(Ph. | Y5(Ph. | Y5(Ph. | Y5(Ph. | Y5(Ph. | Y5(Ph. | Y5(Ph. |
| | | | Eur) | Eur) | Eur) | Eur) | Eur) | Eur) | Eur) | Eur) | Eur) |
| | Visible pparticle | | No | No | No | 1 white | No | No | 1 white | No | No |
| | | | obvious | obvious | obvious | visible | obvious | obvious | visible | obvious | obvious |
| | | | visible | visible | visible | fibrous | visible | visible | fibrous | visible | visible |
| | | | particle | particle | particle | particlesmaller | particle | particle | particle | particle | particle |
| | | | | | | than 2 mm | | | smaller | | |
| | | | | | | | | | than 2 mm | | |
| | Moisture (%) | | 0.87 | 0.84 | 0.79 | 0.74 | 0.77 | 0.63 | 0.76 | 0.63 | 0.66 |
| | Protein | | 20.7 | 20.5 | 20.5 | 20.5 | 20.3 | 20.2 | 20.4 | 20.7 | 20.6 |
| | concentration(mg/ml) | | | | | | | | | | |
| | pH | | 5.1 | 5.1 | 5.1 | 5.1 | 5.1 | 5.1 | 5.1 | 5.1 | 5.1 |
| | SEC-HPLC (%) | | 98.9 | 98.9 | 98.9 | 99.0 | 99.0 | 99.1 | 99.0 | 99.0 | 99.0 |
| | nrCE-SDS (%) | | 96.5 | 96.0 | 95.9 | 96.2 | 96.2 | 96.3 | 96.2 | 96.2 | 96.2 |
| | rCE-SDS (%) | | 97.2 | 97.2 | 97.2 | 97.3 | 97.3 | 97.3 | 97.3 | 97.2 | 97.3 |
| | CEX- | Acid peak | 31.5 | 31.9 | 31.5 | 30.8 | 31.1 | 31.0 | 31.2 | 31.3 | 31.0 |
| | HPLC | main peak | 56.6 | 56.3 | 56.5 | 57.4 | 57.1 | 57.2 | 57.1 | 57.0 | 57.2 |
| | (%) | basic peak | 11.9 | 11.8 | 12.1 | 11.8 | 11.8 | 11.8 | 11.7 | 11.7 | 11.9 |

| | | | | | | | | | |
|---|---|---|---|---|---|---|---|---|---|
| Free Drug (%) | <0.005 | <0.005 | <0.005 | <0.005 | <0.005 | <0.005 | <0.005 | <0.005 | <0.005 |
| HIC-HPLC | 1.79 | 1.78 | 1.79 | 1.80 | 1.79 | 1.79 | 1.79 | 1.79 | 1.79 |
| (DAR value) | | | | | | | | | |
| Binding activity | 92 | 87 | 92 | 96 | 96 | 91 | 98 | 100 | 97 |
| (%) | | | | | | | | | |
| Biological | 121 | 124 | 121 | 117 | 126 | 123 | 121 | 109 | 124 |
| activity (%) | | | | | | | | | |

### 5.5.5 Formulation Process Development Conclusion

Light for 12 days (5000±500 lx) had no effect on the quality of C19388 freeze-dried drug product. It is recommended to store the drug product in the dark.

The freeze-drying process parameters used in the production of C19388 drug products were:

| | | | Freeze | |
|---|---|---|---|---|
| | | Temperature | drying | |
| | | change | chamber | |
| | Temper- | rate (° C. | pressure | Time |
| Freeze drying stage | ature(° C.) | ° C./min) | (Pa) | (h) |
| Cooling of the pre- | 20~−45 | 1.0 | Normal | 1 |
| freezing | | | pressure | |
| Pre-freezing | −45 | NA | Normal | 4 |
| temperature | | | pressure | |
| Primary drying | −45~−20 | 0.42 | 25 Pa | 1 |
| Primary drying | −20 | NA | 25 Pa | 49 |
| temperature | | | | |
| Secondary drying | −20~30 | 0.42 | 25 Pa | 2 |
| Secondary drying | 30 | NA | 25 Pa | 8 |
| temperature | | | | |

### 5.5.6 Experimental Steps of Batch Number: GQ1001-190902

The preparation process development further confirmed by examining the stability of another batch product. The stability of the product of batch number GQ1001-190902 prepared by the present invention was determined using the same buffer solution and the same method as the product of batch number C19388-20190909 in Experiment 5.5.3. The products were sampled and detected at 0-hour, 5 days, 10 days and 14 days. The testing items and the results are as following table:

TABLE 5-4′
The test results of illumination experiment (Batch: GQ1001-190902)

| | | | Illumination | | | Shading |
|---|---|---|---|---|---|---|
| Testing items | | T0 | 5 d | 10 d | 14 d | 5 d |
| Charge Variant | Main Peak | 58.7% | 58.2% | 57.4% | 56.2% | 59.5% |
| (CEX-HPLC) | Acidic peak | 29.3% | 30.3% | 31.0% | 32.5% | 29.0% |
| | Basic peak | 12.0% | 11.5% | 11.7% | 11.2% | 11.5% |
| Purity (SEC- | Monomer | 99.1% | 99.0% | 98.9% | 98.8% | 99.1% |
| HPLC) | Aggregate | 0.9% | 1.0% | 1.1% | 1.2% | 0.9% |
| | Fragment | 0.0% | 0.0% | 0.0% | 0.0% | 0.0% |
| Purity (Non- | Monomer | 96.9% | 96.1% | 95.9% | 95.8% | 96.9% |
| reduced CE- | Fragment | 3.1% | 3.9% | 4.1% | 4.2% | 3.2% |
| SDS) | | | | | | |
| Purity | LC + HC | 96.8% | 97.2% | 96.6% | 96.4% | 97.1% |
| (Reduced CE- | NGHC (non- | 0.9% | 0.8% | 0.8% | 0.8% | 0.8% |
| SDS) | glycosilation HC) | | | | | |
| Free drug | | <0.005% | <0.005% | <0.005% | <0.005% | <0.005% |
| Biological activity | | 100% | 102% | 106% | 95% | 94% |
| (Cell-Based Assay) | | | | | | |
| Binding activity (ELISA) | | 100% | 96% | 99% | 100% | 100% |
| Protein Content (UV) | | 101 mg/Vial | 100 mg/Vial | 106 mg/Vial | 104 mg/Vial | 100 mg/Vial |
| pH (pH Test) | | 5.0 | 5.1 | 5.1 | 5.1 | 5.1 |
| DAR (HIC- | D0 | 0.7% | 0.7% | 0.6% | 0.7% | 0.7% |
| HPLC) | DAR | 1.81 | 1.81 | 1.80 | 1.80 | 1.81 |
| | | Shading | | Avoid light | | |
| Testing items | | 10 d | 14 d | 5 d | 10 d | 14 d |
| Charge Variant | Main Peak | 59.9% | 59.3% | 59.3% | 59.6% | 58.9% |
| (CEX-HPLC) | Acidic peak | 28.3% | 29.2% | 29.1% | 28.6% | 29.5% |
| | Basic peak | 11.8% | 11.5% | 11.6% | 11.8% | 11.6% |
| Purity (SEC- | Monomer | 99.0% | 99.0% | 99.1% | 99.1% | 99.0% |
| HPLC) | Aggregate | 1.0% | 1.0% | 0.9% | 1.0% | 1.0% |
| | Fragment | 0.0% | 0.0% | 0.0% | 0.0% | 0.0% |
| Purity (Non- | Monomer | 97.0% | 97.0% | 96.8% | 96.8% | 97.0% |
| reduced CE- | Fragment | 3.0% | 3.0% | 3.2% | 3.2% | 3.0% |
| SDS) | | | | | | |
| Purity | LC + HC | 97.1% | 96.9% | 97.2% | 97.3% | 97.0% |
| (Reduced CE- | NGHC (non- | 0.8% | 0.8% | 0.8% | 0.8% | 0.8% |
| SDS) | glycosilation HC) | | | | | |
| Free drug | | <0.005% | <0.005% | <0.005% | <0.005% | <0.005% |
| Biological activity | | 104% | 94% | 99% | 100% | 97% |
| (Cell-Based Assay) | | | | | | |
| Binding activity (ELISA) | | 98% | 103% | 95% | 103% | 100% |
| Protein Content (UV) | | 98 mg/Vial | 101 mg/Vial | 98 mg/Vial | 99 mg/Vial | 101 mg/Vial |
| pH (pH Test) | | 5.1 | 5.1 | 5.1 | 5.1 | 5.1 |
| DAR (HIC- | D0 | 0.6% | 0.7% | 0.7% | 0.6% | 0.6% |
| HPLC) | DAR | 1.81 | 1.81 | 1.81 | 1.81 | 1.81 |

According to the results above, compared with the sample at 0-hour, when the protein was placed in the light (5000±500 lx) for 14 days, protein content, pH, and DAR value had no significant changes, and the biological activity and binding activity were within 100±10%, the purity of SEC-HPLC, the purity of CE-SDS, and the proportion of main peak of CEX-HPLC did not decrease significantly, and the free drug also did not change significantly. The similar results were obtained in the condition of avoiding light and shading, that is, the protein stability is quite good. Therefore, lighting for 14 days (5000±500 lx) has no effect on the quality of GQ1001-190902, and the light stability is quite good.

1. A freeze-drying process of an ADC formulation, comprising the following steps:
(1) Pre-freezing:
transferring the ADC formulation to the freezer layer of a lyophilizer,
lowering the temperature with a cooling rate of about 0.36° C./min or more;
(2) Primary drying:
raising the temperature and drying under the pressure of about 30 Pa or less;
(3) Secondary drying:
raising the temperature to about 20-40° C.;
wherein,
step (1) comprises a cooling process and a holding process;
step (2) comprises a heating process and a holding process;
step (3) comprises a heating process and a holding process;
wherein,
the ADC is GQ1001 having the structure of 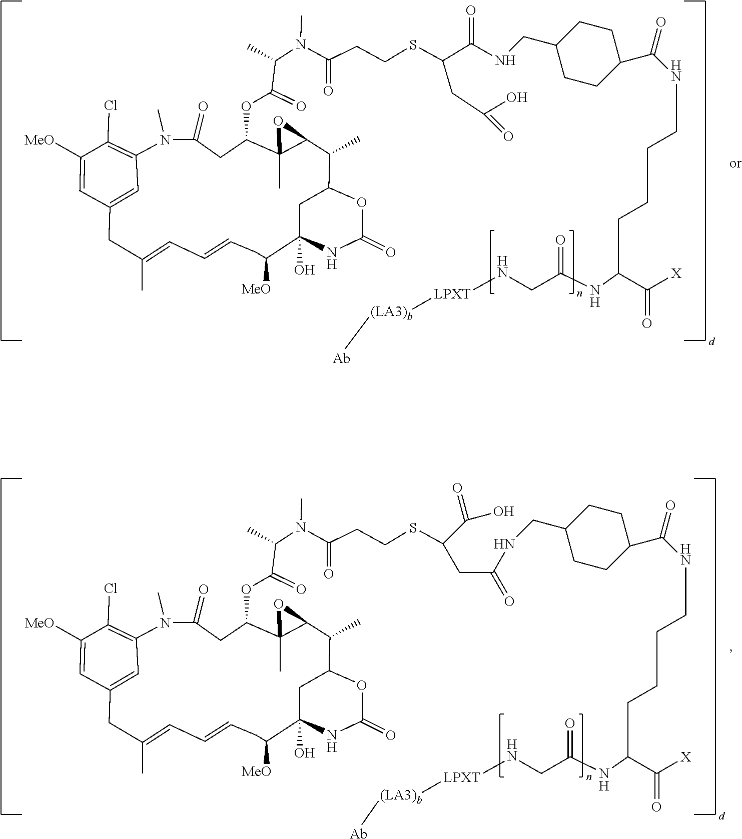
wherein n is 3, d is 2, the X in the ligase recognition sequence LPXT is a glutamic acid (E), Ab is Trastuzumab, LA3 is linker moiety, comprising 1 to 100 series-connected structure units which are selected from the group consisting of one or more glycine and alanine; each b is independently 0 or 1, indicating the presence or absence of LA3; x is —OH or —NH₂ group; preferably, the ADC is 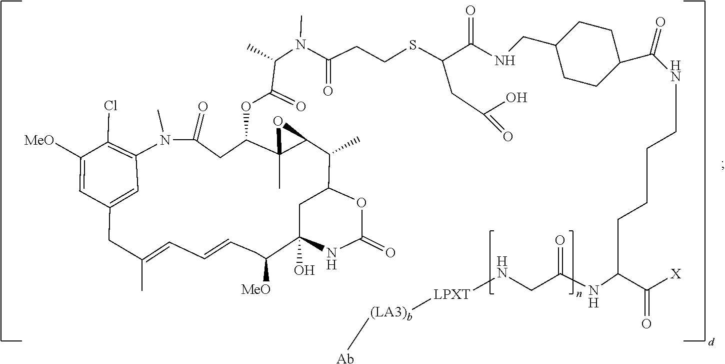
wherein,
the ADC formulation comprises
15-25 mg/ml GQ1001,
8-12 mmol/L sodium succinate,
4-8% sucrose (W/V),
0.01-0.03% polysorbate 20 (W/V), and
pH is 4.8-5.2.

2. The freeze-drying process of claim 1, wherein the ADC formulation comprises
20 mg/ml GQ1001,
10 mmol/L sodium succinate,
6% sucrose (W/V),
0.02% polysorbate 20 (W/V), and
pH is 5.0.

3. The freeze-drying process of claim 1, wherein the cooling rate of the cooling process in step (1) is no more than about 2° C./min; the pressure of the freeze-drying chamber during step (2) and step (3) is no less than about 20 Pa.

4. The freeze-drying process of claim 1, wherein the initial temperature of ADC formulation in step (1) is about 20° C. and the temperature of the holding process in step (1) is −45° C. or less, preferably −45° C.; the holding time in step (1) is 4 hours or more, preferably 4 hours, and the pressure of the freeze-drying chamber during the holding process in step (1) is normal pressure.

5. The freeze-drying process of claim 1, wherein the cooling time in step (1) is 3 hour or less, preferably 1 hour; the cooling rate is 0.5° C./min or more, preferably 1° C./min; the pressure of the freeze-drying chamber during the cooling process in step (1) is normal pressure.

6. The freeze-drying process of claim 1, wherein the increase temperature rate of the heating process in step (2) is 5-26° C./h, preferably 6-25° C./h, more preferably 6° C./h or 25° C./h; the increase temperature time of the heating process in step (2) is 5 hours or less, preferably 4 hours or 1 hour; the pressure of the heating process in step (2) is 20-30 Pa, preferably 25 Pa.

7. The freeze-drying process of claim 1, wherein the temperature of holding process in step (2) is −26 to −18° C., preferably −25 to −20° C., more preferably −25° C. or −20° C., most preferably −20° C.; the holding time in step (2) is 10-55 hours, preferably 40-49 hours, more preferably 43 hours or 49 hours; the pressure of the freeze-drying chamber during the holding process in step (2) is 20-30 Pa, preferably 25 Pa.

8. The freeze-drying process of claim 1, wherein the increase temperature rate of the heating process in step (3) is 10-30° C./h, preferably 17° C./h or 25° C./h, the increase temperature time in step (3) is 2-6 hours, preferably 2-4 hours, more preferably 2 hours or 3 hours; the pressure of the freeze-drying chamber during the heating process in step (3) is 20-30 Pa, preferably 25 Pa.

9. The freeze-drying process of claim 1, wherein the temperature of the holding process in step (3) is 20-35° C., preferably 25-30° C., more preferably 25° C. or 30° C., most preferably 30° C.; the holding time in step (3) is 6 hours or more, preferably 8 hours; the pressure of the freeze-drying chamber during the holding process in step (3) is 20-30 Pa, preferably 25 Pa.

10. A The freeze-drying process of claim 1, which is used for a full-tank freeze-drying process.
